SEQUENCE LISTING (9 sequences)

SEQ ID NO: 1 - DNA, 1437 bp, Homo sapiens
atggcgtcacgcatagggttgcgcatgcagctcatgcgggagcaggcgcagcaggaggagcagcgggagcgcatgcagcaacaggctgtcatgcattacatgcagcagcagcagcagcagcaacagcagcagctcggagggccgcccaccccggccatcaatacccccgtccacttccagtcgccaccacctgtgcctggggaggtgttgaaggtgcagtcctacctggagaatcccacatcctaccatctgcagcagtcgcagcatcagaaggtgcgggagtacctgtccgagacctatgggaacaagtttgctgcccacatcagcccagcccagggctctccgaaacccccaccagccgcctccccaggggtgcgagctggacacgtgctgtcctcctccgctggcaacagtgctcccaatagccccatggccatgctgcacattggctccaaccctgagagggagttggatgatgtcattgacaacattatgcgtctggacgatgtccttggctacatcaatcctgaaatgcagatgcccaacacgctacccctgtccagcagccacctgaatgtgtacagcagcgacccccaggtcacagcctccctggtgggcgtcaccagcagctcctgccctgcggacctgacccagaagcgagagctcacagatgctgagagcagggccctggccaaggagcggcagaagaaagacaatcacaacttaattgaaaggagacgaaggttcaacatcaatgaccgcatcaaggagttgggaatgctgatccccaaggccaatgacctggacgtgcgctggaacaagggcaccatcctcaaggcctctgtggattacatccggaggatgcagaaggacctgcaaaagtccagggagctggagaaccactctcgccgcctggagatgaccaacaagcagctctggctccgtatccaggagctggagatgcaggctcgagtgcacggcctccctaccacctccccgtccggcatgaacatggctgagctggcccagcaggtggtgaagcaggagctgcctagcgaagagggcccaggggaggccctgatgctgggggctgaggtccctgaccctgagccactgccagctctgcccccgcaagccccgctgcccctgcccacccagccaccatccccattccatcacctggacttcagccacagcctgagctttgggggcagggaggacgagggtcccccgggctaccccgaacccctggcgccggggcatggctccccattccccagcctgtccaagaaggatctggacctcatgctcctggacgactcactgctaccgctggcctctgatccacttctgtccaccatgtcccccgaggcctccaaggccagcagccgccggagcagcttcagcatggaggagggcgatgtgctgtgagaattc

SEQ ID NO: 2 - protein, 476 aa, Homo sapiens
MASRIGLRMQLMREQAQQEEQRERMQQQAVMHYMQQQQQQQQQQLGGPPTPAINTPVHFQSPPPVPGEVLKVQSYLENPTSYHLQQSQHQKVREYLSETYGNKFAAHISPAQGSPKPPPAASPGVRAGHVLSSSAGNSAPNSPMAMLHIGSNPERELDDVIDNIMRLDDVLGYINPEMQMPNTLPLSSSHLNVYSSDPQVTASLVGVTSSSCPADLTQKRELTDAESRALAKERQKKDNHNLIERRRRFNINDRIKELGMLIPKANDLDVRWNKGTILKASVDYIRRMQKDLQKSRELENHSRRLEMTNKQLWLRIQELEMQARVHGLPTTSPSGMNMAELAQQVVKQELPSEEGPGEALMLGAEVPDPEPLPALPPQAPLPLPTQPPSPFHHLDFSHSLSFGGREDEGPPGYPEPLAPGHGSPFPSLSKKDLDLMLLDDSLLPLASDPLLSTMSPEASKASSRRSSFSMEEGDVL

SEQ ID NO: 3 - DNA, 1409 bp, Homo sapiens
ctagcaattagctagctgggaaagggctgggccccatgtaaatatttctaaagcacccctctcccctcccccctcagatcaggagtctgagggagaggcacagaggctccctttctctaagccagtcctcacctgcctaagaagatgtgaaggagacccaggagaccctgggatagggaggaactcagagggaagggacattcttttcttcgtcgcaatcctgggagctccctggaggaggagacccgatcagcctgcaatcctggcgcgtcccaggaggagaaagcggcttcctctatactgtactctcctccacagaacccccctctcagccctggaagtccttgctcacagccgaggcgccgagagcgcttgctctgcccagatctgcgcgagtctggcgcccgcgctctgaacggcgtcgctgcccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctcggccgcc

SEQ ID NO: 4 - DNA, 2147 bp, Homo sapiens
ctttggggagtcctaagagggcagctggcaatggacacctagcagtccctttgagacttatttcagatggagctgtagaaagatgccatggctcacagtgcctccctgggaagggggcagagggctgcccagtgaggcctcttgcgagcaggaaatcaccagagacaaggaaagaccagaccccaggatgacctcagttaggccttgcccgactgtcctcagagtcccattctctgtgtcctggttcttttagaagatcatggacctccaggtcatttcgtaaccggaatctgcctgcggggggttttgacaagctatggtatagtgtatgtgggggtactgacgaattggaagatcatggagaccccttctcctcctccatcattggtctgccacatccctcccaggcgactcacagcagagagaccttggatgtatgtagggtgctttaaaactccagctgagttacagtctctcctttctgttttcaccttaaccttccagggatgcaaacccacgacaggtttagcagcagagtggaggctggccatgaatctcagagaaagtgctcactggaaaggctggtttagcccaggcctgatgtggaggcactgagctggacgttctagcggggttgacacccaacagtttacatagggggaggccacccctcctgagcagtctcggtgacttgaagaggaagccgcttcttctgtaccaacacagaagctccagcgaacccccagaatgctggcagtgtgggtgctatgtaaaagtatttacatagctttgtagagtgagccaagcccagtctgtttgggatgactcttcacagtgcctcgaatctgtcacacgtcttagtaagcagagtcacagagtttctgtcacatcatcctcctgcctacagggaagtaggccatgtccctgccccctactctgagcccagctgtgggagccagccctgcccaatgggctctctctgattggcttctcactcacttctaaactccagtgagcaacttctctcggctcgttcaattggcgtgaaggtctgtgtcttgcagagaaggttcttcacaactgggataaaggtctcgctgctcaagtgtagcccagtagaactgccaagccccttcccctcctctccctagactcttggatgcaagaagaatccaggcagctccaagggtgattgtgtccaacctagaatgtcttgaaaaagacattaaggggactagagaagacaggggatccaacggttctctgcagcccagcctgactgacatgtaactcttctggttctcaccagccagctggacctgcttagtattctttctgcctcagtttcccagcctgtacccagggctgtcatagttccatttcaggcagtagtaatgaatgagctgacataaaacatttagagcaggggtcagtatgtatatagagtgattattctatatcaggcattgcctcctcggaatgaagcttacaatcacccctccctctgcagttcatcttggggtggccagaggatccagcagacacctagtggggtaacacaccccagccaactcggctgttgcagactttgtctagaagtttcacgtctcagagctgaattcccttctcatgacctttggccgtgggagtgacacctcacagctgtggtgttttgacaaccagcagccactggcacacaaaatgtgcagccagcagcatatgaagtccaagaggcgtcccggccagccctgtccttgacccccacctgacaattaaggcaagagcctatagtttgcatcagcaacagtcacggtcaaagtttagtcaatcaaacgttgtgtaaggactcaactatggctgacacgggggcctgaggcctcccaacattcattaacaacagcaagttcaatcattatctccccaaagtttattgtgttaggtcagttccaaaccgtgctgaccatggctatgatccaaaggccggccccttacgtcagaggcgagcctccaggtccagctgaggggcagggctgtcctcccttctgtatactatttaaagcgaggagggctagctaccaagcacggttggccttccctctgggaacacacccttggccaacaggggaaatccggcgagacgctctgag

SEQ ID NO: 5 - DNA, 1601 bp, Mus musculus
atggctcagctcgctcagtggtcttgggcaaatcccttctgcccggactcagtttctccttgtgcacaatgggagcaaccatacttatgccagcctgtgcttaaagactacgaagatgatgaatacttcatgggcctgtctcccctcgactacagggagcccgaaccaacagctgccatggcgtcacgcatcgggctgcgcatgcagctcatgcgggagcaggcccagcaggaggagcagcgagagcgcatgcagcagcaggctgtcatgcattatatgcaacagcagcagcagcagcagcagcagctgggtgggccccccaccccagccatcaacacccctgtccacttccagtcgcccccgcctgtgcccggggaggtgctgaaggtgcagtcctacctggagaaccccacctcctaccacctgcaacagtcccagcatcagaaggttcgggagtatctgtctgagacctatgggaacaagtttgctgcccacgtgagcccagcccaaggttccccgaagcctgccccagcagcatccccaggggtgcgggctggacacgtactgtccacctcggccggcaacagtgctcccaacagtcccatggccatgctacatatcagctccaaccccgagaaagagtttgatgatgtcattgacaacattatgcgcctggacagcgtgctgggctacatcaaccctgagatgcagatgcctaacacgctgcccctgtctagcagccacctgaacgtgtacagcggtgacccccaggtcacagcctccatggtgggtgtcaccagcagctcctgccctgccgacctgactcagaagcgagagctaacagatgctgagagcagagccctggccaaggagcggcagaagaaagacaatcacaacctaattgagagaagacgcaggttcaacatcaatgaccggatcaaggagctgggaatgctgatccccaaggccaacgacctggacgtgcgctggaacaaaggcaccatcctcaaggcctctgtggattacatccggaggatgcagaaggacctgcagaagtcccgggagctggagaaccactcccggcgcctggagatgactaacaagcagctctggctccgcatccaggagctggagatgcaggcacgcgtgcacggcctccccaccacctcgccgtcgggtgtgaatatggccgagctggcccagcaggtggtgaagcaagagttgcccagtgaggatggcccaggggaggcgctgatgctggggcctgaggtccctgagcctgagcaaatgccggctcttcctccccaggctccgctgccctcggccgcccagccacagtctccgttccatcacctggacttcagccatggcctgagctttgggggtgggggcgacgaggggcccacaggttaccccgataccctggggacagagcacggctccccattccccaacctgtccaagaaggatctggacttaatgctcctagatgactccctgctccccctggcctctgaccccctcttttctaccatgtctcctgaggcctccaaggccagcagccgccggagcagcttcagcatggaggagggtgatgttct

SEQ ID NO: 6 - DNA, 6468 bp, Artificial Sequence
  pAAV2.1 CMV-mTFEB plasmid
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcatggctcagctcgctcagtggtcttgggcaaatcccttctgcccggactcagtttctccttgtgcacaatgggagcaaccatacttatgccagcctgtgcttaaagactacgaagatgatgaatacttcatgggcctgtctcccctcgactacagggagcccgaaccaacagctgccatggcgtcacgcatcgggctgcgcatgcagctcatgcgggagcaggcccagcaggaggagcagcgagagcgcatgcagcagcaggctgtcatgcattatatgcaacagcagcagcagcagcagcagcagctgggtgggccccccaccccagccatcaacacccctgtccacttccagtcgcccccgcctgtgcccggggaggtgctgaaggtgcagtcctacctggagaaccccacctcctaccacctgcaacagtcccagcatcagaaggttcgggagtatctgtctgagacctatgggaacaagtttgctgcccacgtgagcccagcccaaggttccccgaagcctgccccagcagcatccccaggggtgcgggctggacacgtactgtccacctcggccggcaacagtgctcccaacagtcccatggccatgctacatatcagctccaaccccgagaaagagtttgatgatgtcattgacaacattatgcgcctggacagcgtgctgggctacatcaaccctgagatgcagatgcctaacacgctgcccctgtctagcagccacctgaacgtgtacagcggtgacccccaggtcacagcctccatggtgggtgtcaccagcagctcctgccctgccgacctgactcagaagcgagagctaacagatgctgagagcagagccctggccaaggagcggcagaagaaagacaatcacaacctaattgagagaagacgcaggttcaacatcaatgaccggatcaaggagctgggaatgctgatccccaaggccaacgacctggacgtgcgctggaacaaaggcaccatcctcaaggcctctgtggattacatccggaggatgcagaaggacctgcagaagtcccgggagctggagaaccactcccggcgcctggagatgactaacaagcagctctggctccgcatccaggagctggagatgcaggcacgcgtgcacggcctccccaccacctcgccgtcgggtgtgaatatggccgagctggcccagcaggtggtgaagcaagagttgcccagtgaggatggcccaggggaggcgctgatgctggggcctgaggtccctgagcctgagcaaatgccggctcttcctccccaggctccgctgccctcggccgcccagccacagtctccgttccatcacctggacttcagccatggcctgagctttgggggtgggggcgacgaggggcccacaggttaccccgataccctggggacagagcacggctccccattccccaacctgtccaagaaggatctggacttaatgctcctagatgactccctgctccccctggcctctgaccccctcttttctaccatgtctcctgaggcctccaaggccagcagccgccggagcagcttcagcatggaggagggtgatgttctgggatcccgggctgactacaaagaccatgacggtgattataaagatcatgacatcgactacaaggatgacgatgacaagtagtgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 7 - DNA, 3657 bp, Artificial Sequence
  pAAV2.1 CMV-mTFEB plasmid from 3′ITR to 5′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcatggctcagctcgctcagtggtcttgggcaaatcccttctgcccggactcagtttctccttgtgcacaatgggagcaaccatacttatgccagcctgtgcttaaagactacgaagatgatgaatacttcatgggcctgtctcccctcgactacagggagcccgaaccaacagctgccatggcgtcacgcatcgggctgcgcatgcagctcatgcgggagcaggcccagcaggaggagcagcgagagcgcatgcagcagcaggctgtcatgcattatatgcaacagcagcagcagcagcagcagcagctgggtgggccccccaccccagccatcaacacccctgtccacttccagtcgcccccgcctgtgcccggggaggtgctgaaggtgcagtcctacctggagaaccccacctcctaccacctgcaacagtcccagcatcagaaggttcgggagtatctgtctgagacctatgggaacaagtttgctgcccacgtgagcccagcccaaggttccccgaagcctgccccagcagcatccccaggggtgcgggctggacacgtactgtccacctcggccggcaacagtgctcccaacagtcccatggccatgctacatatcagctccaaccccgagaaagagtttgatgatgtcattgacaacattatgcgcctggacagcgtgctgggctacatcaaccctgagatgcagatgcctaacacgctgcccctgtctagcagccacctgaacgtgtacagcggtgacccccaggtcacagcctccatggtgggtgtcaccagcagctcctgccctgccgacctgactcagaagcgagagctaacagatgctgagagcagagccctggccaaggagcggcagaagaaagacaatcacaacctaattgagagaagacgcaggttcaacatcaatgaccggatcaaggagctgggaatgctgatccccaaggccaacgacctggacgtgcgctggaacaaaggcaccatcctcaaggcctctgtggattacatccggaggatgcagaaggacctgcagaagtcccgggagctggagaaccactcccggcgcctggagatgactaacaagcagctctggctccgcatccaggagctggagatgcaggcacgcgtgcacggcctccccaccacctcgccgtcgggtgtgaatatggccgagctggcccagcaggtggtgaagcaagagttgcccagtgaggatggcccaggggaggcgctgatgctggggcctgaggtccctgagcctgagcaaatgccggctcttcctccccaggctccgctgccctcggccgcccagccacagtctccgttccatcacctggacttcagccatggcctgagctttgggggtgggggcgacgaggggcccacaggttaccccgataccctggggacagagcacggctccccattccccaacctgtccaagaaggatctggacttaatgctcctagatgactccctgctccccctggcctctgaccccctcttttctaccatgtctcctgaggcctccaaggccagcagccgccggagcagcttcagcatggaggagggtgatgttctgggatcccgggctgactacaaagaccatgacggtgattataaagatcatgacatcgactacaaggatgacgatgacaagtagtgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 23 bp, Artificial Sequence
  Synthetic primer
gcagaagaaagacaatcacaacc

SEQ ID NO: 9 - DNA, 18 bp, Artificial Sequence
  Synthetic primer
gccttggggatcagcatt

(19) United States
(12) Patent Application Publication
(10) Pub. No.: US 2015/0273016 A1
(43) Pub. Date: Oct. 1, 2015

(54) GENE THERAPY FOR GLYCOGEN STORAGE DISEASES

(71) Applicant: FONDAZIONE TELETHON, Roma (IT)

(72) Inventors: Giancarlo Parenti, Napoli (NA) (IT); Andrea Ballabio, Napoli (NA) (IT)

(21) Appl. No.: 14/436,161

(22) PCT Filed: Oct. 17, 2013
(86) PCT No.: PCT/EP2013/003131

§ 371 Date: Apr. 16, 2015

Related U.S. Application Data
(60) Provisional application No. 61/715,187, filed on Oct. 17, 2012.

(51) Int. Cl. A61K 38/17; C12N 15/86

(52) U.S. Cl. CPC A61K 38/1709; C12N 15/86; A61K 48/00

(57) ABSTRACT

The present invention relates to a transcription factor EB (TFEB) protein, ortholog, recombinant or synthetic or bio technological functional derivative thereof, allelic variant thereof and fragments thereof; a chimeric molecule comprising the TFEB protein, ortholog, recombinant or synthetic or biotechnological functional derivative thereof, allelic variant thereof and fragments thereof; a polynucleotide coding for said protein or ortholog, recombinant or synthetic or biotechnological functional derivative thereof, allelic variant thereof and fragments thereof; a vector comprising said polynucleotide; a host cell genetically engineered expressing said polypeptide or a pharmaceutical composition for use in the treatment or/and prevention of a glycogen storage disease. Preferably of Pompe or Danon disease.

### CROSS-REFERENCE TO RELATED APPLICATIONS

The present application claims the benefit of U.S. Provisional Application No. 61/715,187, filed Oct. 17, 2012, the contents of which are incorporated herein by reference.

### TECHNICAL FIELD

The present invention relates to a transcription factor EB (TFEB) protein, ortholog, recombinant or synthetic or biotechnological functional derivative thereof, allelic variant thereof and fragments thereof; a chimeric molecule comprising the TFEB protein, ortholog, recombinant or synthetic or biotechnological functional derivative thereof, allelic variant thereof and fragments thereof; a polynucleotide coding for said protein or ortholog, recombinant or synthetic or biotechnological functional derivative thereof, allelic variant thereof and fragments thereof; a vector comprising said polynucleotide; a host cell genetically engineered expressing said polypeptide or a pharmaceutical composition for use in the treatment or/and prevention of a glycogen storage disease. Preferably of Pompe or Danon disease.

### BACKGROUND OF THE INVENTION

A number of therapeutic approaches have been explored for the treatment of the lysosomal storage diseases (LSD), based on different strategies and rationale. These include hematopoietic stem cell transplantation (HSCT), enzyme replacement therapy (ERT), substrate reduction therapy (SRT), pharmacological chaperone therapy (PCT), and gene therapy (GT).

Generally speaking, these approaches can be divided into two broad categories: those that are aimed at increasing the residual activity of the missing enzyme (such as HSCT, ERT, PCT and GT), and those that are directed toward reducing the synthesis of the accumulated substrate(s) (SRT). Typically, the goal of these therapies is to restore the equilibrium of the so called “storage equation”, that is the balance between the synthesis and the degradation of substrates.

However, each of these approaches is typically indicated only for specific diseases, and many of them do not allow complete cure of the various aspects of multisystem disorders like LSDs.

Pompe disease is a lysosomal storage disease, also belonging to the group of glycogen storage diseases, caused by a deficiency or dysfunction of the lysosomal hydrolase acid alpha-glucosidase (GAA), a glycogen-degrading lysosomal enzyme. Deficiency of GAA results in lysosomal glycogen accumulation in many tissues in Pompe patients, with cardiac and skeletal muscle tissues most seriously affected. The combined incidence of all forms of Pompe disease is estimated to be 1:40,000, and the disease affects all groups without an ethnic predilection. It is estimated that approximately one third of those with Pompe disease have the rapidly progressive, fatal infantile-onset form, while the majority of patients present with the more slowly progressive, juvenile or late-onset forms.

At present the only approved treatment for Pompe disease, enzyme replacement therapy, has shown important limitations (Schoser et al. (2008) “Therapeutic approaches in glycogen storage disease type II/Pompe Disease,” Neurotherapeutics 5:569-7). For example, despite treatment, some patients have limited clinical benefit, in particular, in skeletal muscles, or show signs of disease progression.

Danon disease is also a glycogen storage disease caused by mutations in a lysosomal enzyme, specifically the LAMP2 gene, which encodes for an essential component of the lysosomal membrane and appears to play a role in autophagosome-lysosome fusion. Danon disease is characterized by severe cardiomyopathy and variable degrees of muscle weakness, frequently associated with intellectual deficit. There is no specific treatment for this disease.

Pompe disease and Danon disease belong to the group of Glycogen storage diseases (GSD, also glycogenosis and dextrinosis). GSDs are due to defects in the processing of glycogen synthesis or breakdown within muscles, liver, and other cell types. GSDs have two classes of cause: genetic and acquired. Genetic GSDs are caused by any inborn error of metabolism (genetically defective enzymes) involved in these processes. Overall, according to a study in British Columbia, approximately 2.3 children per 100000 births (1 in 43,000) have some form of glycogen storage disease. In the United States, they are estimated to occur in 1 per 20000-25000 births. A Dutch study estimated it to be 1 in 40000.

### SUMMARY OF THE INVENTION

The present invention provides improved therapy for glycogen storage diseases, in particular Pompe or Danon disease. In part, the present invention is based on the discovery that transcription factor EB (TFEB), member of the basic-helix-loop-helix leucine-zipper transcription factor MiTF/TFE family, can be effectively delivered to skeletal muscles using gene therapy approach to induce lysosomal exocytosis and discharge of accumulated storage material into the extracellular space, resulting in effective clearance of glycogen storages in muscles and amelioration of muscular pathology. Thus, the present invention prove for the first time that the clearance of accumulated substrates induced by TFEB over-expression in main diseased tissues such as skeletal muscles may cure the clinical manifestations of glycogen storage diseases, in particular Pompe or Danon disease.

It is therefore an object of the present invention a compound selected in the group consisting of:

a) a transcription factor EB (TFEB) protein, ortholog, recombinant or synthetic or biotechnological functional derivative thereof, allelic variant thereof and fragments thereof;
b) a chimeric molecule comprising the TFEB protein, ortholog, recombinant or synthetic or biotechnological functional derivative thereof, allelic variant thereof and fragments thereof;
c) a polynucleotide coding for said protein or ortholog, recombinant or synthetic or biotechnological functional derivative thereof, allelic variant thereof and fragments thereof;
d) a vector comprising said polynucleotide;
e) a host cell genetically engineered expressing said polypeptide
for use in the treatment or/and prevention of a glycogen storage disease.

Preferably, the glycogen storage disease is characterized by accumulation of glycogen in muscle, liver, heart, and/or nervous system.

Still preferably, the glycogen storage disease is selected from the group consisting of: GSD type Ia (Von Gierke disease), GSD type I non-a (various subtypes), GSD type II (Pompe disease), GSD type IIb (Danon disease), GSD type III (Cori's disease or Forbes' disease), GSD type IV (Andersen disease), GSD type V (McArdle disease), GSD type VI (Hers' disease), GSD type VII (Tarui's disease), GSD type IX, GSD type XI (Fanconi-Bickel syndrome), GSD type XII (Red cell aldolase deficiency), GSD type XIII and GSD type 0.

Yet preferably the glycogen storage disease is Pompe disease or Danon disease.

Preferably, the compound is delivered to a target tissue that contains accumulated glycogen. Preferably, the target tissue is selected from muscle, liver, heart, and/or nervous system. Preferably, the target tissue is muscle and/or liver. Still preferably, the muscle is skeletal muscle, cardiac muscle, and/or diaphragm. Yet preferably the compound is delivered by systemic administration. Preferably the systemic administration is an intravenous administration.

In a preferred embodiment, the compound is delivered by local administration. Preferably, the local administration is an intramuscular administration. In a preferred embodiment the TFEB protein comprises an amino acid sequence at least 80% identical to SEQ ID NO:2.

Preferably the TFEB protein comprises an amino acid sequence at least 90% identical to SEQ ID NO:2. Still preferably the TFEB protein comprises an amino acid sequence consisting of SEQ ID NO:2.

In a preferred embodiment the polynucleotide comprises a tissue specific promoter sequence that controls the expression of the TFEB protein.

Preferably, the tissue specific promoter sequence is a muscle specific promoter sequence, preferably it is the MCK promoter sequence consisting of SEQ ID NO: 3:

CTAGCAATTAGCTAGCTGGGAAAGGGCTGGGCCCCATGTAAATATTTCTA
AAGCACCCCTCTCCCCTCCCCCCTCAGATCAGGAGTCTGAGGGAGAGGCA
CAGAGGCTCCCTTTCTCTAAGCCAGTCCTCACCTGCCTAAGAAGATGTGA
AGGAGACCCAGGAGACCCTGGGATAGGGAGGAACTCAGAGGGAAGGGACA
TTCTTTTCTTCGTCGCAATCCTGGGAGCTCCCTGGAGGAGGAGACCCGAT
CAGCCTGCAATCCTGGCGCGTCCCAGGAGGAGAAAGCGGCTTCCTCTATA
CTGTACTCTCCTCCACAGAACCCCCCTCTCAGCCCTGGAAGTCCTTGCTC
ACAGCCGAGGCGCCGAGAGCGCTTGCTCTGCCCAGATCTGCGCGAGTCTG
GCGCCCGCGCTCTGAACGGCGTCGCTGCCCAGCCCCCTTCCCCGGGAGGT
GGGAGCGGCCACCCAGGGCCCCGTGGCTGCCCTTGTAAGGAGGCGAGGCC
CGAGGACACCCGAGACGCCCGGTTATAATTAACCAGGACACGTGGCGAAC
CCCCCTCCAACACCTGCCCCCGAACCCCCCCATACCCAGCGCCTCGGGTC
TCGGCCTTTGCGGCAGAGGAGACAGCAAAGCGCCCTCTAAAAATAACTCC
TTTCCCGGCGACCGAGACCCTCCCTGTCCCCCGCACAGCGGAAATCTCCC
AGTGGCACCGAGGGGGCGAGGGTTAAGTGGGGGGGAGGGTGACCACCGCC
TCCCACCCTTGCCCTGAGTTTGAATCTCTCCAACTCAGCCAGCCTCAGTT
TCCCCTCCACTCAGTCCCTAGGAGGAAGGGGCGCCCAAGCGCGGGTTTCT
GGGGTTAGACTGCCCTCCATTGCAATTGGTCCTTCTCCCGGCCTCTGCTT
CCTCCAGCTCACAGGGTATCTGCTCCTCCTGGAGCCACACCTTGGTTCCC
CGAGGTGCCGCTGGGACTCGGGTAGGGGTGAGGGCCCAGGGGGCACAGGG
GGAGCCGAGGGCCACAGGAAGGGCTGGTGGCTGAAGGAGACTCAGGGGCC
AGGGGACGGTGGCTTCTACGTGCTTGGGACGTTCCCAGCCACCGTCCCAT
GTTCCCGGCGGGGGGCCAGCTGTCCCCACCGCCAGCCCAACTCAGCACTT
GGTCAGGGTATCAGCTTGGTGGGGGGGCGTGAGCCCAGCCCCTGGGGCGG
CTCAGCCCATACAAGGCCATGGGGCTGGGCGCAAAGCATGCCTGGGTTCA
GGGTGGGTATGGTGCGGGAGCAGGGAGGTGAGAGGCTCAGCTGCCCTCCA
GAACTCCTCCCTGGGGACAACCCCTCCCAGCCAATAGCACAGCCTAGGTC
CCCCTATATAAGGCCACGGCTGCTGGCCCTTCCTTTGGGTCAGTGTCACC
TCGGCCGCC
(from Tessitore A. et al, Mol Ther, 2007)

Preferably the tissue specific promoter sequence is a liver specific promoter sequence, preferably it is the PEPCK promoter sequence consisting of SEQ ID NO: 4

CTTTGGGGAGTCCTAAGAGGGCAGCTGGCAATGGACACCTAGCAGTCCCT
TTGAGACTTATTTCAGATGGAGCTGTAGAAAGATGCCATGGCTCACAGTG
CCTCCCTGGGAAGGGGGCAGAGGGCTGCCCAGTGAGGCCTCTTGCGAGCA
GGAAATCACCAGAGACAAGGAAAGACCAGACCCCAGGATGACCTCAGTTA
GGCCTTGCCCGACTGTCCTCAGAGTCCCATTCTCTGTGTCCTGGTTCTTT
TAGAAGATCATGGACCTCCAGGTCATTTCGTAACCGGAATCTGCCTGCGG
GGGGTTTTGACAAGCTATGGTATAGTGTATGTGGGGGTACTGACGAATTG
GAAGATCATGGAGACCCCTTCTCCTCCTCCATCATTGGTCTGCCACATCC
CTCCCAGGCGACTCACAGCAGAGAGACCTTGGATGTATGTAGGGTGCTTT
AAAACTCCAGCTGAGTTACAGTCTCTCCTTTCTGTTTTCACCTTAACCTT
CCAGGGATGCAAACCCACGACAGGTTTAGCAGCAGAGTGGAGGCTGGCCA
TGAATCTCAGAGAAAGTGCTCACTGGAAAGGCTGGTTTAGCCCAGGCCTG
ATGTGGAGGCACTGAGCTGGACGTTCTAGCGGGGTTGACACCCAACAGTT
TACATAGGGGGAGGCCACCCCTCCTGAGCAGTCTCGGTGACTTGAAGAGG
AAGCCGCTTCTTCTGTACCAACACAGAAGCTCCAGCGAACCCCCAGAATG
CTGGCAGTGTGGGTGCTATGTAAAAGTATTTACATAGCTTTGTAGAGTGA
GCCAAGCCCAGTCTGTTTGGGATGACTCTTCACAGTGCCTCGAATCTGTC
ACACGTCTTAGTAAGCAGAGTCACAGAGTTTCTGTCACATCATCCTCCTG
CCTACAGGGAAGTAGGCCATGTCCCTGCCCCCTACTCTGAGCCCAGCTGT
GGGAGCCAGCCCTGCCCAATGGGCTCTCTCTGATTGGCTTCTCACTCACT
TCTAAACTCCAGTGAGCAACTTCTCTCGGCTCGTTCAATTGGCGTGAAGG
TCTGTGTCTTGCAGAGAAGGTTCTTCACAACTGGGATAAAGGTCTCGCTG
CTCAAGTGTAGCCCAGTAGAACTGCCAAGCCCCTTCCCCTCCTCTCCCTA
GACTCTTGGATGCAAGAAGAATCCAGGCAGCTCCAAGGGTGATTGTGTCC
AACCTAGAATGTCTTGAAAAAGACATTAAGGGGACTAGAGAAGACAGGGG
ATCCAACGGTTCTCTGCAGCCCAGCCTGACTGACATGTAACTCTTCTGGT
TCTCACCAGCCAGCTGGACCTGCTTAGTATTCTTTCTGCCTCAGTTTCCC
AGCCTGTACCCAGGGCTGTCATAGTTCCATTTCAGGCAGTAGTAATGAAT
GAGCTGACATAAAACATTTAGAGCAGGGGTCAGTATGTATATAGAGTGAT
TATTCTATATCAGGCATTGCCTCCTCGGAATGAAGCTTACAATCACCCCT
CCCTCTGCAGTTCATCTTGGGGTGGCCAGAGGATCCAGCAGACACCTAGT
GGGGTAACACACCCCAGCCAACTCGGCTGTTGCAGACTTTGTCTAGAAGT
TTCACGTCTCAGAGCTGAATTCCCTTCTCATGACCTTTGGCCGTGGGAGT
GACACCTCACAGCTGTGGTGTTTTGACAACCAGCAGCCACTGGCACACAA
AATGTGCAGCCAGCAGCATATGAAGTCCAAGAGGCGTCCCGGCCAGCCCT
GTCCTTGACCCCCACCTGACAATTAAGGCAAGAGCCTATAGTTTGCATCA
GCAACAGTCACGGTCAAAGTTTAGTCAATCAAACGTTGTGTAAGGACTCA
ACTATGGCTGACACGGGGGCCTGAGGCCTCCCAACATTCATTAACAACAG
CAAGTTCAATCATTATCTCCCCAAAGTTTATTGTGTTAGGTCAGTTCCAA
ACCGTGCTGACCATGGCTATGATCCAAAGGCCGGCCCCTTACGTCAGAGG
CGAGCCTCCAGGTCCAGCTGAGGGGCAGGGCTGTCCTCCCTTCTGTATAC
TATTTAAAGCGAGGAGGGCTAGCTACCAAGCACGGTTGGCCTTCCCTCTG
GGAACACACCCTTGGCCAACAGGGGAAATCCGGCGAGACGCTCTGAG

In a preferred embodiment, the polynucleotide comprises a nucleotide sequence at least 60% identical to SEQ 1D NO:1. Preferably, the polynucleotide comprises a nucleotide sequence at least 80% identical to SEQ ID NO:1. More preferably the polynucleotide comprises a nucleotide sequence consisting of SEQ ID NO:1.

In a preferred embodiment, the vector is an expression vector selected in the group consisting of: viral vector, plasmids, viral particles and phages.

Preferably the viral vector is selected from the group consisting of: adenoviral vectors, lentiviral vectors, retroviral vectors, adeno associated vectors (AAV) and naked plasmid DNA vectors.

Preferably the AAV vector is selected from the group consisting of AAV1, AAV2, AAV5, AAV6, AAV7, AAV8, AAV9, and combination thereof.

More preferably, the AAV vector is an AAV1, AAV2 or AAV9 vector.

Still preferably the AAV vector is a chimeric and/or pseudotyped vector.

Preferably, the delivery of said molecule results in reduced storage of glycogen in muscles and/or liver. Preferably the muscles are skeletal muscles.

More preferably, the delivery of said molecule results in reduced storage of glycogen in muscles and/or liver in terms of intensity, severity, or frequency, or has delayed onset.

It is a further object of the invention a pharmaceutical composition for use in the treatment and/or prevention of a glycogen storage disease comprising a pharmaceutically acceptable excipient and a compound as defined in any one of the preceding claims.

It is a further object of the invention a method of treating a glycogen storage disease comprising a step of delivering a nucleic acid encoding a transcription factor EB (TFEB) gene into a subject in need of treatment.

In the method preferably the nucleic acid encoding the TFEB gene is delivered to a target tissue that contains accumulated glycogen. More preferably the target tissue is selected from muscle, liver, heart, and/or nervous system. Still preferably the target tissue is muscle and/or liver. More preferably the muscle is skeletal muscle, cardiac muscle, and/or diaphragm. In the method preferably the nucleic acid is delivered by systemic administration. Preferably the systemic administration is intravenous administration. Preferably the nucleic acid is delivered by local administration. More preferably the local administration is an intramuscular administration. Still preferably wherein the nucleic acid is a viral vector. Preferably the viral vector is an adeno-associated virus (AAV) vector.

Still preferably the AAV vector is selected from the group consisting of AAV1, AAV2, AAV5, AAV6, AAV7, AAV8, AAV9, and combination thereof. Yet preferably the AAV vector is an AAV1, AAV2 or AAV9 vector. More preferably the AAV vector is a chimeric and/or pseudotyped vector.

In a preferred embodiment, the nucleic acid further comprises a tissue specific promoter sequence that controls the expression of the TFEB genes.

Preferably, the tissue specific promoter sequence is a muscle specific promoter sequence, preferably it is the MCK promoter sequence consisting of SEQ ID NO: 3.

Preferably, the tissue specific promoter sequence is a liver specific promoter sequence, preferably it is the PEPCK promoter sequence consisting of SEQ ID NO: 4.

In the method preferably the TFEB gene comprises a nucleotide sequence at least 60% identical to SEQ ID NO:1. Preferably, the TFEB gene comprises a nucleotide sequence at least 80% identical to SEQ ID NO:1. Still preferably the TFEB gene comprises a nucleotide sequence of SEQ ID NO:1. Preferably, the TFEB gene comprises a nucleotide sequence encoding an amino acid sequence at least 80% identical to SEQ ID NO:2.

In a preferred embodiment, the TFEB gene comprises a nucleotide sequence encoding an amino acid sequence at least 90% identical to SEQ ID NO:2.

Preferably the TFEB gene comprises a nucleotide sequence encoding an amino acid sequence of SEQ ID NO:2.

Preferably the delivery of the nucleic acid encoding the TFEB gene results in reduced storage of glycogen in muscles and/or liver. Still preferably the delivery of the nucleic acid encoding the TFEB gene results in reduced storage of glycogen in skeletal muscles.

It is a further object of the invention a method of treating a glycogen storage disease comprising a step of administering a nucleic acid encoding a transcription factor EB (TFEB) gene into a subject in need of treatment such that the glycogen storage in muscles and/or liver is reduced in intensity, severity, or frequency, or has delayed onset.

Preferably, the nucleic acid encoding the TFEB gene is administered systemically. Preferably, the TFEB gene is administered intravenously.

In a preferred embodiment the nucleic acid encoding the TFEB gene is administered intramuscularly.

More preferably the nucleic acid is an expression vector selected in the group consisting of: viral vector, plasmids, viral particles and phages.

Preferably, the viral vector is selected from the group consisting of: adenoviral vectors, lentiviral vectors, retroviral vectors, adeno associated vectors (AAV) and naked plasmid DNA vectors.

Preferably, the AAV vector is selected from the group consisting of AAV1, AAV2, AAV5, AAV6, AAV7, AAV8, AAV9, and combination thereof.

Still preferably, the AAV vector is an AAV1, AAV2 or AAV9 vector.

In particular embodiments, the target tissue is muscle (e.g., skeletal muscle, cardiac muscle and/or diaphragm).

In the present invention a recombinant, synthetic or biotechnological functional derivative, allelic variant of a protein, peptide fragments of a protein, chimeric molecules comprising the TFEB protein, synthetic or biotechnological functional derivative thereof are defined as molecules able to maintain the therapeutic effect of TFEB, i.e the treatment of glycogen storage diseases, in particular Pompe or Danon disease.

The derivatives are selected in the group comprising proteins having a percentage of identity of at least 45%, preferably at least 75%, more preferably of at least 85%, still preferably of at least 90% or 95% with SEQ ID NO. 2 or orthologs thereof.

Fragments refer to proteins having a length of at least 50 amino acids, preferably at least 100 amino acids, more preferably at least 150 amino acids.

The polynucleotide of the invention is selected in the group consisting of RNA or DNA, preferably said polynucleotide is DNA.

In the present invention the host cell is selected in the group consisting of: bacterial cells, fungal cells, insect cells, animal cells, and plant cells, preferably said host cells is an animal cell.

The pharmaceutical composition is for systemic, oral or topical administration.

In the present invention, the viral vector may be selected from the group of: adenoviral vectors, adeno-associated viral (AAV) vectors, pseudotyped AAV vectors, herpes viral vectors, retroviral vectors, lentiviral vectors, baculoviral vectors. Pseudotyped AAV vectors are vectors which contain the genome of one AAV serotype in the capsid of a second AAV serotype; for example an AAV2/8 vector contains the AAV8 capsid and the AAV 2 genome (Auricchio et al. Hum. Mol. Genet. 10(26):3075-81 (2001)). Such vectors are also known as chimeric vectors. Naked plasmid DNA vectors and other vectors known in the art may be used to deliver a TFEB gene according to the present invention³⁶. Other examples of delivery systems include ex vivo delivery systems, which include but are not limited to DNA transfection methods such as electroporation, DNA biolistics, lipid-mediated transfection, compacted DNA-mediated transfection. Typically, a viral vector can accommodate a transgene (i.e., a TFEB gene described herein) and regulatory elements. Various methods may be used to deliver viral vectors encoding a TFEB gene described herein into a subject in need of treatment. For example, a viral vector may be delivered through intravenous or intravascular injection. Other routes of systemic administration include, but are not limited to, intra-arterial, intra-cardiac, intraperitoneal and subcutaneous or via local administration such as muscle injection or intramuscular administration.

The vector of the invention, in particular an AAV vector, in particular AAV2/1 or AAV2/9 vectors may be injected at a dose range between 1×10e10 viral particles (vp)/kg and 1×10e13 vp/kg.

A dose range between 1×10e11 and 1×10e12 vp/kg is more likely to be effective in humans because these doses are expected to result in large transduction efficiency of muscle (heart and skeletal muscle) and liver.

The vector of the invention, in particular the AAV vector may be injected at doses between 1×10e11 vector genomes (vg)/kg and 1×10e13 vg/kg are expected to provide high muscle and liver transduction (Nathwani, A. C., et al. N Engl J Med 365, 2357-2365 (2011)).

Adenoviral vector genomes do not integrate into the genome of the transduced cells and therefore vector genomes are lost in actively dividing cells³⁷. Should TFEB expression fade over time, to maintain phenotypic correction it would be possible to re-administer a vector with a different serotype to overcome the neutralizing anti-Ad antibody elicited with the first administration ((Kim et al. Proc Natl. Acad Sci USA 98: 13282-13287 (2001); Morral et al. Proc Natl Acad Sci USA. 1999; 96:12816-12821) (1999)).

The present invention provides pharmaceutical compositions comprising: a) an effective amount of a vector as described herein or an effective amount of a transformed host cell as described herein, and b) a pharmaceutically acceptable carrier, which may be inert or physiologically active. As used herein, “pharmaceutically-acceptable carriers or excipients” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH ˜7.4, containing or not containing about 1 mg/ml to 25 mg/ml human serum albumin, (2) 0.9% saline (0.9% w/v sodium chloride (NaCl)), and (3) 5% (w/v) dextrose; and may also contain an antioxidant such as tryptamine and a stabilizing agent such as Tween 20.

The pharmaceutical compositions encompassed by the present invention may also contain a further therapeutic agent for the treatment of glycogen storage diseases, in particular Pompe or Danon disease.

The compositions of the invention may be in a variety of forms. These include for example liquid, semi-solid, but the preferred form depends on the intended mode of administration and therapeutic application. Typical preferred compositions are in the form of injectable or infusible solutions. The preferred mode of administration is parenteral (e.g. intravenous, intramuscular, intraperinoneal, subcutaneous). In a preferred embodiment, the compositions of the invention are administered intravenously as a bolus or by continuous infusion over a period of time. In another preferred embodiment, they are injected by intramuscular, subcutaneous, intraarticular, intrasynovial, intratumoral, peritumoral, intralesional, or perilesional routes, to exert local as well as systemic therapeutic effects.

Sterile compositions for parenteral administration can be prepared by incorporating the vector or host cell as described in the present invention in the required amount in the appropriate solvent, followed by sterilization by micro filtration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohols, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

There may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The doses depend on the desired effect, the duration of the treatment and the route of administration used and may be determined easily by the skilled person in the art using known methods.

As well known in the medical arts, dosages for any one patient depends upon many factors, including the patient's size, body surface area, age, the particular compound to be administered, sex, time and route of administration, general health, and other drugs being administered concurrently.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The Figures described below, that together make up the Drawings, are for illustration purposes only, not for limitation.

FIG. 1 shows schematic representation of the AAV2.1-cytomegalovirus (CMV) plasmid containing the murine Tcfeb coding sequence (mTFEB).

FIG. 2 illustrates exemplary results showing promotion of glycogen clearance and attenuation of Pompe Disease (PD) pathology in α-glucosidase (GAA)−/− mice injected intramuscularly with AAV2/1-CMV-mTFEB. (A) Glycogen assay in TFEB-injected gastrocnemii and in contralateral untreated muscles showing significant decrease in glycogen levels in TFEB-injected muscles as compared to the untreated muscles. (B) Period acid Schiff (PAS) staining of TFEB-treated gastrocnemii showing reduction in punctate staining corresponding to lysosomal glycogen stores, compared to untreated muscles. (C) Lysosomal-associated membrane protein 1 (LAMP1) staining of TFEB-injected gastrocnemii and contralateral untreated muscles showing a reduction in the number and size of the LAMP1 vesicles in TFEB treated muscles as compared to untreated muscles.

FIG. 3 depicts exemplary electron microscopy (EM) analysis of the impact of TFEB-injection on the muscle fiber ultrastructure in GAA−/− mice. (A, B) Asterisks (*) in low magnification images of muscle fibers illustrate lysosome-like organelles containing glycogen.

(C, D) Measurement of length of lysosomes (average±SE; n=100 lysosomal structures) and their number in 5 μm² area of muscle fiber section (average±SE; n=50 fields). (E, F) Asterisks (*) in high magnification images of lysosome-like organelles reveal looser pattern of glycogen in their lumen upon TFEB overexpression. Black arrows indicate autophagosome profiles; white arrow shows remnants of mitochondria engulfed into the lysosome interior. (G) Measurement of number of autophagosomes flanking the glycogen-containing lysosome-like structures (average±SD; n=100 lysosomes). “***” in panels C, D, and G indicates statistically significant differences according to t-test with p<0.001. Scale bars: 1500 nm in A and B; 450 nm in E and F.

FIG. 4 illustrates behavioral tests (wire hanging, steel hanging and rotarod) in wild-type mice, GAA−/− untreated knockout mice, and GAA−/− AAV2/9-CMV-mTFEB-treated animals. Both GAA−/− untreated and GAA−/− TFEB-treated mice showed impaired performance at the hanging wire (A), hanging steel (B), and rotarod (C) tests, compared to wild-type animals. However, in all tests TFEB-treated animals showed a trend towards improved performance, compared to untreated animals.

FIG. 5 illustrates TFEB expression levels analyzed by real-time PCR in liver (A) and gastrocnemii (B) of AAV2/9-CMV-mTFEB-treated mice. In the treated animals the analysis showed an increase of approximately 4 fold in liver and of approximately 2 fold in gastrocnemius, compared to their relative controls.

FIG. 6 illustrates Glycogen levels in gastrocnemii from untreated and AAV2/9-CMV-mTFEB-treated GAA−/− mice (Gaa−/−). In TFEB-treated animals glycogen levels were lower compared to untreated animals.

### DETAILED DESCRIPTION

The present invention provides, nucleic acids molecules, vectors, methods and compositions for treating glycogen storage diseases, in particular Pompe or Danon disease, based on over-expression of transcription factor EB (TFEB) gene in target tissues, such as, muscles using gene therapy approach. In particular, the present invention provides a method of treating Pompe disease by delivering a nucleic acid encoding a TFEB gene into a subject in need of treatment. In some embodiments, a nucleic acid encoding a TFEB gene is delivered by systemic administration (e.g., intravenous administration). In some embodiments, a suitable TFEB gene is delivered by a viral vector, such as, an adeno-associated virus (AAV) vector.

Various aspects of the invention are described in detail in the following sections. The use of sections is not meant to limit the invention. Each section can apply to any aspect of the invention. In this application, the use of “or” means “and/or” unless stated otherwise.

### Glycogen Storage Diseases

Glycogen storage diseases (GSD, also glycogenosis and dextrinosis) are the result of defects in the processing of glycogen synthesis or breakdown within muscles, liver, and other cell types. GSDs may be genetic or acquired, and are characterized by abnormal inherited glycogen metabolism in the liver, muscle and brain. Genetic GSDs are caused by inborn error of metabolisms and involve genetically defective enzymes. They are mostly inherited as autosomal recessive disorders and result in defects of glycogen synthesis or catabolism. The overall incidence of GSDs is estimated at 1 case per 20000-40000 live births. Disorders of glycogen degradation may affect primarily the liver, the muscle or both. There are over 12 types and they are classified based on the enzyme deficiency and the affected tissue. (Mingyi Chen, Glycogen Storage Diseases, Molecular Pathology Library, Volume 5, 2011, pp 677-681)

GSDs include the following types and related subtypes:

| Type or Synonym | Defective enzyme or transporter |
|---|---|
| GSD type Ia (Von Gierke disease) | glucose-6-phosphatase |
| GSD type I non-a (various | Glucose-6-phosphate translocase |
| subtypes) | and other defective proteins with |
| | unknown function |
| GSD type II (Pompe disease) | acid alpha-glucosidase |
| GSD type IIb (Danon disease) | Lysosomal-associated membrane |
| | protein 2 |
| GSD type III (Cori's disease or | glycogen debranching enzyme |
| Forbes' disease) | |
| GSD type IV (Andersen disease) | glycogen branching enzyme |
| GSD type V (McArdle disease) | muscle glycogen phosphorylase |
| GSD type VI (Hers' disease) | liver glycogen phosphorylase |
| GSD type VII (Tarui's disease) | muscle phosphofructokinase |
| GSD type IX | phosphorylase kinase |
| GSD type XI (Fanconi-Bickel | glucose transporter |
| syndrome) | |
| GSD type XII (Red cell aldolase | Aldolase A |
| deficiency) | |
| GSD type XIII | β-enolase |
| GSD type 0 | glycogen synthase |

Pompe disease is a rare genetic disorder caused by a deficiency in the enzyme acid alpha-glucosidase (GAA), which is needed to break down glycogen, a stored form of sugar used for energy. Pompe disease is also known as glycogen storage disease type II, GSD 11, type 11 glycogen storage disease, glycogenosis type 11, acid maltase deficiency, alpha-1,4-glucosidase deficiency, cardiomegalia glycogenic diffusa, and cardiac form of generalized glycogenosis. The build-up of glycogen causes progressive muscle weakness (myopathy) throughout the body and affects various body tissues, particularly in the heart, skeletal muscles, liver, respiratory and nervous system.

The presenting clinical manifestations of Pompe disease can vary widely depending on the age of disease onset and residual GAA activity. Residual GAA activity correlates with both the amount and tissue distribution of glycogen accumulation as well as the severity of the disease. Infantile-onset Pompe disease (less than 1% of normal GAA activity) is the most severe form and is characterized by hypotonia, generalized muscle weakness, and hypertrophic cardiomyopathy, and massive glycogen accumulation in cardiac and other muscle tissues. Death usually occurs within one year of birth due to cardiorespiratory failure (Hirschhorn et al. (2001) “Glycogen Storage Disease Type II: Acid Alpha-glucosidase (Acid Maltase) Deficiency,” in Scriver et al., eds., The Metabolic and Molecular Basis of Inherited Disease, 8th Ed., New York: McGraw-Hill, 3389-3420). Juvenile-onset (1-10% of normal GAA activity) and adult-onset (10-40% of normal GAA activity) Pompe disease are more clinically heterogeneous, with greater variation in age of onset, clinical presentation, and disease progression. Juvenile- and adult-onset Pompe disease are generally characterized by lack of severe cardiac involvement, later age of onset, and slower disease progression, but eventual respiratory or limb muscle involvement results in significant morbidity and mortality. While life expectancy can vary, death generally occurs due to respiratory failure (Hirschhorn et al. (2001) “Glycogen Storage Disease Type II: Acid Alpha-glucosidase (Acid Maltase) Deficiency,” in Scriver et al., eds., The Metabolic and Molecular Basis of Inherited Disease, 8th Ed., New York: McGraw-Hill, 3389-3420).

Danon disease (glycogen storage disease Type IIb or glycogen storage disease with normal acid maltase) is a metabolic disorder originally described by Danon et al. characterized clinically by severe cardiomyopathy and variable degrees of muscle weakness, frequently associated with intellectual deficit. The pathological hallmark of the disease is intracytoplasmic vacuoles containing autophagic material and glycogen in skeletal and cardiac muscle cells. The disease classically manifests in males over 10 years of age. The clinical picture may be severe in both sexes, but onset generally occurs later in females. The disease is transmitted as an X-linked recessive trait and is caused by mutations in the LAMP2 gene, localised to Xq24. The LAMP2 protein is an essential component of the lysosomal membrane and appears to play a role in autophagosome-lysosome fusion. Biological diagnosis revolves around demonstration of normal or high acid maltase activity in combination with muscle biopsies showing large vacuoles (filled with glycogen and products of cytoplasmic degradation) and an absence of the LAMP-2 protein on immunohistochemical analysis. The diagnosis can be confirmed by molecular analysis of the LAMP2 gene. The differential diagnosis should include X-linked myopathy with excessive autophagia (XMEA) and Pompe disease. There is no specific treatment for this disease. Symptomatic treatment is required for the cardiac manifestations and patients may require a heart transplant. Patients are at risk of sudden death due to arrhythmia during early adulthood. (Nishino 1, Fu J, Tanji K, Yamada T, Shimojo S, Koori T, Mora M, Riggs J E, Oh S J, Koga Y, Sue C M, Yamamoto A, Murakami N, Shanske S, Byrne E, Bonilla E, Nonaka I, DiMauro S, Hirano M. Primary LAMP-2 deficiency causes X-linked vacuolar cardiomyopathy and myopathy (Danon disease). Nature. 2000 Aug. 24; 406(6798):906-10; Sugie K, Yamamoto A, Murayama K, Oh S J, Takahashi M, Mora M, Riggs J E, Colomer J, Iturriaga C, Meloni A, Lamperti C, Saitoh S, Byrne E, DiMauro S, Nonaka I, Hirano M, Nishino 1. Clinicopathological features of genetically confirmed Danon disease. Neurology. 2002 Jun. 25; 58(12):1773-8).

### Transcription Factor EB

Transcription factor EB (TFEB) is a bHLH-leucine zipper transcription factor. TFEB is a master regulator for a group of genes involved in lysosomal biogenesis [Coordinated Lysosomal Expression and Regulation (CLEAR) network] (Sardiello et al. (2009) “A gene network regulating lysosomal biogenesis and function”, Science 325(5939):473-7; Palmieri et al. (2011) “Characterization of the CLEAR network reveals an integrated control of cellular clearance pathway”, Hum Mol Genet. 20(19):3852-66). In addition, TFEB regulates the biogenesis of autophagosomes by controlling the expression of multiple genes along the autophagic pathway (Settembre and Ballabio (2011) “TFEB regulates autophagy: an integrated coordination of cellular degradation and recycling processes”, Autophagy 7(11):1379-81; Settembre et al. (2011) “TFEB links autophagy to lysosomal biogenesis”, Science 332(6036):1429-33).

In some embodiments, a TFEB gene suitable for the present invention comprises a nucleotide sequence as shown in SEQ ID NO:1

### HUMAN TFEB, NCBI GeneID=7942; Nt=NM_{—}007162.2, Protein=NP_{—}009093.1 (Aa. 1-476)

(SEQ ID NO: 1)
ATGGCGTCACGCATAGGGTTGCGCATGCAGCTCATGCGGGAGCAGGCGCA
GCAGGAGGAGCAGCGGGAGCGCATGCAGCAACAGGCTGTCATGCATTACA
TGCAGCAGCAGCAGCAGCAGCAACAGCAGCAGCTCGGAGGGCCGCCCACC
CCGGCCATCAATACCCCCGTCCACTTCCAGTCGCCACCACCTGTGCCTGG
GGAGGTGTTGAAGGTGCAGTCCTACCTGGAGAATCCCACATCCTACCATC
TGCAGCAGTCGCAGCATCAGAAGGTGCGGGAGTACCTGTCCGAGACCTAT
GGGAACAAGTTTGCTGCCCACATCAGCCCAGCCCAGGGCTCTCCGAAACC
CCCACCAGCCGCCTCCCCAGGGGTGCGAGCTGGACACGTGCTGTCCTCCT
CCGCTGGCAACAGTGCTCCCAATAGCCCCATGGCCATGCTGCACATTGGC
TCCAACCCTGAGAGGGAGTTGGATGATGTCATTGACAACATTATGCGTCT
GGACGATGTCCTTGGCTACATCAATCCTGAAATGCAGATGCCCAACACGC
TACCCCTGTCCAGCAGCCACCTGAATGTGTACAGCAGCGACCCCCAGGTC
ACAGCCTCCCTGGTGGGCGTCACCAGCAGCTCCTGCCCTGCGGACCTGAC
CCAGAAGCGAGAGCTCACAGATGCTGAGAGCAGGGCCCTGGCCAAGGAGC
GGCAGAAGAAAGACAATCACAACTTAATTGAAAGGAGACGAAGGTTCAAC
ATCAATGACCGCATCAAGGAGTTGGGAATGCTGATCCCCAAGGCCAATGA
CCTGGACGTGCGCTGGAACAAGGGCACCATCCTCAAGGCCTCTGTGGATT
ACATCCGGAGGATGCAGAAGGACCTGCAAAAGTCCAGGGAGCTGGAGAAC
CACTCTCGCCGCCTGGAGATGACCAACAAGCAGCTCTGGCTCCGTATCCA
GGAGCTGGAGATGCAGGCTCGAGTGCACGGCCTCCCTACCACCTCCCCGT
CCGGCATGAACATGGCTGAGCTGGCCCAGCAGGTGGTGAAGCAGGAGCTG
CCTAGCGAAGAGGGCCCAGGGGAGGCCCTGATGCTGGGGGCTGAGGTCCC
TGACCCTGAGCCACTGCCAGCTCTGCCCCCGCAAGCCCCGCTGCCCCTGC
CCACCCAGCCACCATCCCCATTCCATCACCTGGACTTCAGCCACAGCCTG
AGCTTTGGGGGCAGGGAGGACGAGGGTCCCCCGGGCTACCCCGAACCCCT
GGCGCCGGGGCATGGCTCCCCATTCCCCAGCCTGTCCAAGAAGGATCTGG
ACCTCATGCTCCTGGACGACTCACTGCTACCGCTGGCCTCTGATCCACTT
CTGTCCACCATGTCCCCCGAGGCCTCCAAGGCCAGCAGCCGCCGGAGCAG
CTTCAGCATGGAGGAGGGCGATGTGCTGTGAGAATTC

In some embodiments, a TFEB gene suitable for the present invention comprises a nucleotide sequence that is substantially identical to SEQ ID NO:1. For example, a suitable TFEB gene may have a nucleotide sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:1.

In some embodiments, a TFEB gene suitable for the present invention comprises a nucleotide sequence encoding an amino acid sequence as shown in SEQ ID N0:2.

(SEQ ID NO: 2)
MASRIGLRMQLMREQAQQEEQRERMQQQAVMHYMQQQQQQQQQQLGGPPT
PAINTPVHFQSPPPVPGEVLKVQSYLENPTSYHLQQSQHQKVREYLSETY
GNKFAAHISPAQGSPKPPPAASPGVRAGHVLSSSAGNSAPNSPMAMLHIG
SNPERELDDVIDNIMRLDDVLGYINPEMQMPNTLPLSSSHLNVYSSDPQV
TASLVGVTSSSCPADLTQKRELTDAESRALAKERQKKDNHNLIERRRRFN
INDRIKELGMLIPKANDLDVRWNKGTILKASVDYIRRMQKDLQKSRELEN
HSRRLEMTNKQLWLRIQELEMQARVHGLPTTSPSGMNMAELAQQVVKQEL
PSEEGPGEALMLGAEVPDPEPLPALPPQAPLPLPTQPPSPFHHLDFSHSL
SFGGREDEGPPGYPEPLAPGHGSPFPSLSKKDLDLMLLDDSLLPLASDPL
LSTMSPEASKASSRRSSFSMEEGDVL.

In some embodiments, a TFEB gene suitable for the present invention comprises a nucleotide sequence encoding an amino acid sequence substantially homologous or identical to SEQ ID N0:2. For example, a suitable TFEB gene may comprise a nucleotide sequence encoding an amino acid sequence at least 50%, 55%, 60%, 65%, 70%, 75%, 80%, 85%, 90%, 91%, 92%, 93%, 94%, 95%, 96%, 97%, 98%, 99% or more homologous or identical to SEQ ID N0:2. In some embodiments, a suitable TFEB gene may comprise a nucleotide sequence encoding a TFEB protein containing amino acid substitutions, deletions and/or insertions. For example, a suitable TFEB gene may comprise a nucleotide sequence encoding a TFEB protein containing mutations at position(s) corresponding to S142 and/or 5211 of human wild type TFEB protein. In particular, a suitable TFEB gene may comprise a nucleotide sequence encoding a TFEB protein containing S142A and/or S211A substitutions.

“Percent (%) nucleic acid or amino acid sequence identity” with respect to the nucleotide or amino acid sequences identified herein is defined as the percentage of nucleotides or amino acids in a candidate sequence that are identical with the nucleotides or amino acids in the reference sequence, after aligning the sequences and introducing gaps, if necessary, to achieve the maximum percent sequence identity. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al. (1990) “Basic local alignment search tool”, J. Mol. Biol. 215(3): 403-410; Altschul et al., Methods in Enzymology; Altschul et al. (1997) Nucleic Acids Res. 25:3389-3402; Baxevanis et al. (1998) Bioinformatics: A Practical Guide to the Analysis of Genes and Proteins, Wiley; and Misener et al. (eds.), Bioinformatics Methods and Protocols (Methods in Molecular Biology, Vol. 132), Humana Press, 1999. In addition to identifying identical sequences, the programs mentioned above typically provide an indication of the degree of identity.

Homologues or analogues of TFEB proteins can be prepared according to methods for altering polypeptide sequence known to one of ordinary skill in the art such as are found in references that compile such methods. In some embodiments, conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In some embodiments, a “conservative amino acid substitution” refers to an amino acid substitution that does not alter the relative charge or size characteristics of the protein in which the amino acid substitution is made.

### Gene Therapy

Various gene therapy vectors may be used to practice the present invention.

In some embodiments, adeno-associated virus (AAV) of any serotype can be used. The serotype of the viral vector used in certain embodiments of the invention is selected from the group consisting from AAV1, AAV2, AAV3, AAV4, AAV5, AAV6, AAV7, AAV8, and AAV9 (see, e.g., Gao et al. (2002) PNAS, 99:11854-11859; and Viral Vectors for Gene Therapy: Methods and Protocols, ed. Machida, Humana Press, 2003). Other serotype besides those listed herein can be used. Furthermore, pseudotyped AAV vectors may also be utilized in the methods described herein. Pseudotyped AAV vectors are those which contain the genome of one AAV serotype in the capsid of a second AAV serotype; for example, an AAV vector that contains the AAV2 capsid and the AAV1 genome or an AAV vector that contains the AAV5 capsid and the AAV 2 genome (Auricchio et al. (2001) Hum. Mol. Genet. 10(26):3075-81). Additional exemplary AAV vectors are recombinant pseudotyped AAV2/1, AAV2/2, AAV2/5, AAV2/7, AAV2/8 and AAV2/9 serotype vectors. Such vectors are also known as chimeric vectors. For example, an AAV2/1 vector has capsid AAV1 and inverted terminal repeat (ITR) AAV2. An exemplary AAV2/9 vector is described in Medina et al. (2011) “Transcriptional activation of lysosomal exocytosis promotes cellular clearance”, Dev. Cell 21(3):421-30, which is incorporated herein by reference.

Typically, AAV vectors are derived from single-stranded (ss) DNA parvoviruses that are nonpathogenic for mammals (reviewed in Muzyscka (1992) Curr. Top. Microb. Immunol., 158:97-1.29). Briefly, recombinant AAV-based vectors have the rep and cap viral genes that account for 96% of the viral genome removed, leaving the two flanking 145-basepair (bp) inverted terminal repeats (ITRs), which are used to initiate viral DNA replication, packaging and integration.

Typically, an AAV vector can accommodate a transgene (i.e., a TFEB gene described herein) and regulatory element of a length up to about 4.5 kb. In some embodiments, the transgene (i.e., TFEB gene) is under the control of the regulatory element such as a tissue specific or ubiquitous promoter. In some embodiments, a ubiquitous promoter such as a CMV promoter is used to control the expression of a TFEB gene. In some embodiments, a tissue specific promoter such as a muscle, or liver specific promoter is used to control the expression of a TFEB gene. As a non-limiting example, a suitable muscle specific promoter is the human muscle creatine kinase (MCK) promoter and a suitable liver specific promoter is phosphoenolpyruvate carboxykinase (PEPCK) promoter.

In addition, adenoviral vectors, retroviral vectors, lentiviral vectors, SV40, naked plasmid DNA vectors and other vectors known in the art may be used to deliver a TFEB gene according to the present invention.

Various methods may be used to deliver viral vectors encoding a TFEB gene described herein into a subject in need of treatment. In particular, a delivery method suitable for the present invention delivers viral vectors encoding a TFEB gene to various target tissues including, but not limited to, muscles (e.g., skeletal muscles, cardiac muscles, diaphragm, etc.), liver, heart, and nervous system. In some embodiments, a viral vector encoding a TFEB gene is delivered via systemic administration. For example, a viral vector may be delivered through intravenous or intravascular injection. Other routes of systemic administration include, but are not limited to, intra-arterial, intra-cardiac, intraperitoneal and subcutaneous. In some embodiments, a viral vector may be delivered via local administration such as muscle injection or intramuscular administration.

### Treatment of Pompe Disease

The methods of the present invention are effective in treating individuals affected by glycogen storage diseases, in particular individuals affected by infantile-, juvenile- or adult-onset Pompe disease. The terms, “treat” or “treatment,” as used herein, refers to amelioration of one or more symptoms associated with the disease, prevention or delay of the onset of one or more symptoms of the disease, and/or lessening of the severity or frequency of one or more symptoms of the disease. For example, treatment can refer to reduction or clearance of glycogen storage in various affected tissues including but not limited to muscles (e.g., skeletal or cardiac muscles), liver, heart, nervous system; amelioration of muscular pathology; improvement of cardiac status (e.g., increase of end-diastolic and/or end-systolic volumes, or reduction, amelioration or prevention of the progressive cardiomyopathy that is typically found in Pompe disease) or of pulmonary function (e.g., increase in crying vital capacity over baseline capacity, and/or normalization of oxygen desaturation during crying); improvement in neurodevelopment and/or motor skills (e.g., increase in AIMS score); or any combination of these effects. In some embodiment, treatment includes improvement of glycogen clearance, particularly in reverse, reduction or prevention of Pompe disease-associated muscular pathology and/or cardiomyopathy. The terms, “improve,” “increase” or “reduce,” as used herein, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of Pompe disease (either infantile, juvenile or adult-onset) as the individual being treated, who is about the same age as the individual being treated (to ensure that the stages of the disease in the treated individual and the control individual(s) are comparable).

The individual (also referred to as “patient” or “subject”) being treated is an individual (fetus, infant, child, adolescent, or adult human) having Pompe disease (i.e., either infantile-, juvenile-, or adult-onset Pompe disease) or having the potential to develop Pompe disease.

### EXAMPLES

The present invention will be better understood in connection with the following Examples. However, it should be understood that these examples are for illustrative purposes only and are not meant to limit the scope of the invention. Various changes and modifications to the disclosed embodiments will be apparent to those skilled in the art and such changes and modifications including, without limitation, those relating to the methods and/or formulations of the invention may be made without departing from the spirit of the invention and the scope of the appended claims.

### Example 1

### Cloning and Production of Adeno-Associated Virus (AAV) Vector

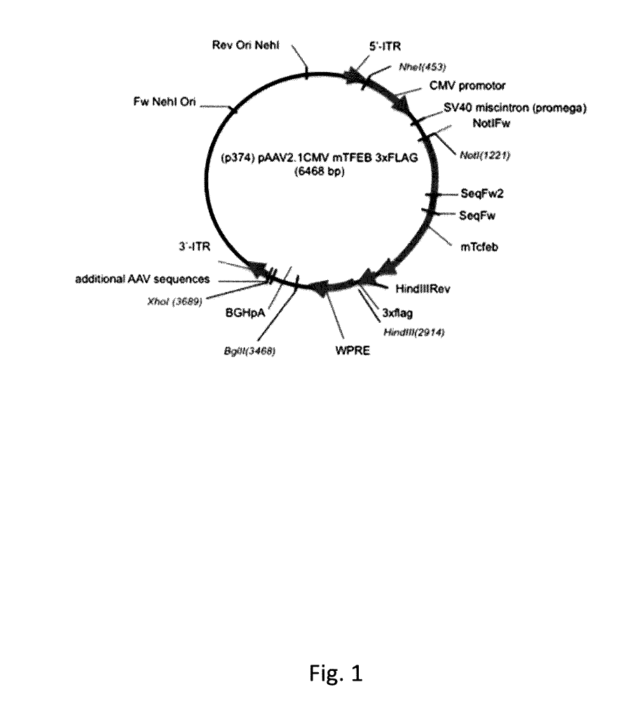

Experiments in this Example illustrate cloning and development of AAV vectors with transcription factor EB gene (FIG. 1).

The coding sequence for murine transcription factor EB, Tcfeb or mTFEB, was cloned into the pAAV2.1-CMV-EGFP plasmid by replacing the EGFP sequence (NotI-HindIII) and fused in frame with a 3× FLAG tag.

mTFEB Sequence (SEQ ID NO: 5):

ATGGCTCAGCTCGCTCAGTGGTCTTGGGCAAATCCCTTCTGCCCGGACTC
AGTTTCTCCTTGTGCACAATGGGAGCAACCATACTTATGCCAGCCTGTGC
TTAAAGACTACGAAGATGATGAATACTTCATGGGCCTGTCTCCCCTCGAC
TACAGGGAGCCCGAACCAACAGCTGCCATGGCGTCACGCATCGGGCTGCG
CATGCAGCTCATGCGGGAGCAGGCCCAGCAGGAGGAGCAGCGAGAGCGCA
TGCAGCAGCAGGCTGTCATGCATTATATGCAACAGCAGCAGCAGCAGCAG
CAGCAGCTGGGTGGGCCCCCCACCCCAGCCATCAACACCCCTGTCCACTT
CCAGTCGCCCCCGCCTGTGCCCGGGGAGGTGCTGAAGGTGCAGTCCTACC
TGGAGAACCCCACCTCCTACCACCTGCAACAGTCCCAGCATCAGAAGGTT
CGGGAGTATCTGTCTGAGACCTATGGGAACAAGTTTGCTGCCCACGTGAG
CCCAGCCCAAGGTTCCCCGAAGCCTGCCCCAGCAGCATCCCCAGGGGTGC
GGGCTGGACACGTACTGTCCACCTCGGCCGGCAACAGTGCTCCCAACAGT
CCCATGGCCATGCTACATATCAGCTCCAACCCCGAGAAAGAGTTTGATGA
TGTCATTGACAACATTATGCGCCTGGACAGCGTGCTGGGCTACATCAACC
CTGAGATGCAGATGCCTAACACGCTGCCCCTGTCTAGCAGCCACCTGAAC
GTGTACAGCGGTGACCCCCAGGTCACAGCCTCCATGGTGGGTGTCACCAG
CAGCTCCTGCCCTGCCGACCTGACTCAGAAGCGAGAGCTAACAGATGCTG
AGAGCAGAGCCCTGGCCAAGGAGCGGCAGAAGAAAGACAATCACAACCTA
ATTGAGAGAAGACGCAGGTTCAACATCAATGACCGGATCAAGGAGCTGGG
AATGCTGATCCCCAAGGCCAACGACCTGGACGTGCGCTGGAACAAAGGCA
CCATCCTCAAGGCCTCTGTGGATTACATCCGGAGGATGCAGAAGGACCTG
CAGAAGTCCCGGGAGCTGGAGAACCACTCCCGGCGCCTGGAGATGACTAA
CAAGCAGCTCTGGCTCCGCATCCAGGAGCTGGAGATGCAGGCACGCGTGC
ACGGCCTCCCCACCACCTCGCCGTCGGGTGTGAATATGGCCGAGCTGGCC
CAGCAGGTGGTGAAGCAAGAGTTGCCCAGTGAGGATGGCCCAGGGGAGGC
GCTGATGCTGGGGCCTGAGGTCCCTGAGCCTGAGCAAATGCCGGCTCTTC
CTCCCCAGGCTCCGCTGCCCTCGGCCGCCCAGCCACAGTCTCCGTTCCAT
CACCTGGACTTCAGCCATGGCCTGAGCTTTGGGGGTGGGGGCGACGAGGG
GCCCACAGGTTACCCCGATACCCTGGGGACAGAGCACGGCTCCCCATTCC
CCAACCTGTCCAAGAAGGATCTGGACTTAATGCTCCTAGATGACTCCCTG
CTCCCCCTGGCCTCTGACCCCCTCTTTTCTACCATGTCTCCTGAGGCCTC
CAAGGCCAGCAGCCGCCGGAGCAGCTTCAGCATGGAGGAGGGTGATGTTC
T

Sequence of the pAAV2.1 CMV-mTFEB Plasmid (SEQ ID NO: 6):

AGCGCCCAATACGCAAACCGCCTCTCCCCGCGCGTTGGCCGATTCATTAA
TGCAGCTGGCACGACAGGTTTCCCGACTGGAAAGCGGGCAGTGAGCGCAA
CGCAATTAATGTGAGTTAGCTCACTCATTAGGCACCCCAGGCTTTACACT
TTATGCTTCCGGCTCGTATGTTGTGTGGAATTGTGAGCGGATAACAATTT
CACACAGGAAACAGCTATGACCATGATTACGCCAGATTTAATTAAGGCTG
CGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAG
TGGCCAACTCCATCACTAGGGGTTCCTTGTAGTTAATGATTAACCCGCCA
TGCTACTTATCTACGTAGCCATGCTCTAGGAAGATCGGAATTCGCCCTTA
AGCTAGCTAGTTATTAATAGTAATCAATTACGGGGTCATTAGTTCATAGC
CCATATATGGAGTTCCGCGTTACATAACTTACGGTAAATGGCCCGCCTGG
CTGACCGCCCAACGACCCCCGCCCATTGACGTCAATAATGACGTATGTTC
CCATAGTAACGCCAATAGGGACTTTCCATTGACGTCAATGGGTGGAGTAT
TTACGGTAAACTGCCCACTTGGCAGTACATCAAGTGTATCATATGCCAAG
TACGCCCCCTATTGACGTCAATGACGGTAAATGGCCCGCCTGGCATTATG
CCCAGTACATGACCTTATGGGACTTTCCTACTTGGCAGTACATCTACGTA
TTAGTCATCGCTATTACCATGGTGATGCGGTTTTGGCAGTACATCAATGG
GCGTGGATAGCGGTTTGACTCACGGGGATTTCCAAGTCTCCACCCCATTG
ACGTCAATGGGAGTTTGTTTTGGCACCAAAATCAACGGGACTTTCCAAAA
TGTCGTAACAACTCCGCCCCATTGACGCAAATGGGCGGTAGGCGTGTACG
GTGGGAGGTCTATATAAGCAGAGCTGGTTTAGTGAACCGTCAGATCCTGC
AGAAGTTGGTCGTGAGGCACTGGGCAGGTAAGTATCAAGGTTACAAGACA
GGTTTAAGGAGACCAATAGAAACTGGGCTTGTCGAGACAGAGAAGACTCT
TGCGTTTCTGATAGGCACCTATTGGTCTTACTGACATCCACTTTGCCTTT
CTCTCCACAGGTGTCCAGGCGGCCGCATGGCTCAGCTCGCTCAGTGGTCT
TGGGCAAATCCCTTCTGCCCGGACTCAGTTTCTCCTTGTGCACAATGGGA
GCAACCATACTTATGCCAGCCTGTGCTTAAAGACTACGAAGATGATGAAT
ACTTCATGGGCCTGTCTCCCCTCGACTACAGGGAGCCCGAACCAACAGCT
GCCATGGCGTCACGCATCGGGCTGCGCATGCAGCTCATGCGGGAGCAGGC
CCAGCAGGAGGAGCAGCGAGAGCGCATGCAGCAGCAGGCTGTCATGCATT
ATATGCAACAGCAGCAGCAGCAGCAGCAGCAGCTGGGTGGGCCCCCCACC
CCAGCCATCAACACCCCTGTCCACTTCCAGTCGCCCCCGCCTGTGCCCGG
GGAGGTGCTGAAGGTGCAGTCCTACCTGGAGAACCCCACCTCCTACCACC
TGCAACAGTCCCAGCATCAGAAGGTTCGGGAGTATCTGTCTGAGACCTAT
GGGAACAAGTTTGCTGCCCACGTGAGCCCAGCCCAAGGTTCCCCGAAGCC
TGCCCCAGCAGCATCCCCAGGGGTGCGGGCTGGACACGTACTGTCCACCT
CGGCCGGCAACAGTGCTCCCAACAGTCCCATGGCCATGCTACATATCAGC
TCCAACCCCGAGAAAGAGTTTGATGATGTCATTGACAACATTATGCGCCT
GGACAGCGTGCTGGGCTACATCAACCCTGAGATGCAGATGCCTAACACGC
TGCCCCTGTCTAGCAGCCACCTGAACGTGTACAGCGGTGACCCCCAGGTC
ACAGCCTCCATGGTGGGTGTCACCAGCAGCTCCTGCCCTGCCGACCTGAC
TCAGAAGCGAGAGCTAACAGATGCTGAGAGCAGAGCCCTGGCCAAGGAGC
GGCAGAAGAAAGACAATCACAACCTAATTGAGAGAAGACGCAGGTTCAAC
ATCAATGACCGGATCAAGGAGCTGGGAATGCTGATCCCCAAGGCCAACGA
CCTGGACGTGCGCTGGAACAAAGGCACCATCCTCAAGGCCTCTGTGGATT
ACATCCGGAGGATGCAGAAGGACCTGCAGAAGTCCCGGGAGCTGGAGAAC
CACTCCCGGCGCCTGGAGATGACTAACAAGCAGCTCTGGCTCCGCATCCA
GGAGCTGGAGATGCAGGCACGCGTGCACGGCCTCCCCACCACCTCGCCGT
CGGGTGTGAATATGGCCGAGCTGGCCCAGCAGGTGGTGAAGCAAGAGTTG
CCCAGTGAGGATGGCCCAGGGGAGGCGCTGATGCTGGGGCCTGAGGTCCC
TGAGCCTGAGCAAATGCCGGCTCTTCCTCCCCAGGCTCCGCTGCCCTCGG
CCGCCCAGCCACAGTCTCCGTTCCATCACCTGGACTTCAGCCATGGCCTG
AGCTTTGGGGGTGGGGGCGACGAGGGGCCCACAGGTTACCCCGATACCCT
GGGGACAGAGCACGGCTCCCCATTCCCCAACCTGTCCAAGAAGGATCTGG
ACTTAATGCTCCTAGATGACTCCCTGCTCCCCCTGGCCTCTGACCCCCTC
TTTTCTACCATGTCTCCTGAGGCCTCCAAGGCCAGCAGCCGCCGGAGCAG
CTTCAGCATGGAGGAGGGTGATGTTCTGGGATCCCGGGCTGACTACAAAG
ACCATGACGGTGATTATAAAGATCATGACATCGACTACAAGGATGACGAT
GACAAGTAGTGAAAGCTTGGATCCAATCAACCTCTGGATTACAAAATTTG
TGAAAGATTGACTGGTATTCTTAACTATGTTGCTCCTTTTACGCTATGTG
GATACGCTGCTTTAATGCCTTTGTATCATGCTATTGCTTCCCGTATGGCT
TTCATTTTCTCCTCCTTGTATAAATCCTGGTTGCTGTCTCTTTATGAGGA
GTTGTGGCCCGTTGTCAGGCAACGTGGCGTGGTGTGCACTGTGTTTGCTG
ACGCAACCCCCACTGGTTGGGGCATTGCCACCACCTGTCAGCTCCTTTCC
GGGACTTTCGCTTTCCCCCTCCCTATTGCCACGGCGGAACTCATCGCCGC
CTGCCTTGCCCGCTGCTGGACAGGGGCTCGGCTGTTGGGCACTGACAATT
CCGTGGTGTTGTCGGGGAAGCTGACGTCCTTTCCATGGCTGCTCGCCTGT
GTTGCCACCTGGATTCTGCGCGGGACGTCCTTCTGCTACGTCCCTTCGGC
CCTCAATCCAGCGGACCTTCCTTCCCGCGGCCTGCTGCCGGCTCTGCGGC
CTCTTCCGCGTCTTCGAGATCTGCCTCGACTGTGCCTTCTAGTTGCCAGC
CATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGG
GGGAGGATTGGGAAGACAATAGCAGGCATGCTGGGGACTCGAGTTAAGGG
CGAATTCCCGATTAGGATCTTCCTAGAGCATGGCTACGTAGATAAGTAGC
ATGGCGGGTTAATCATTAACTACAAGGAACCCCTAGTGATGGAGTTGGCC
ACTCCCTCTCTGCGCGCTCGCTCGCTCACTGAGGCCGGGCGACCAAAGGT
CGCCCGACGCCCGGGCTTTGCCCGGGCGGCCTCAGTGAGCGAGCGAGCGC
GCAGCCTTAATTAACCTAATTCACTGGCCGTCGTTTTACAACGTCGTGAC
TGGGAAAACCCTGGCGTTACCCAACTTAATCGCCTTGCAGCACATCCCCC
TTTCGCCAGCTGGCGTAATAGCGAAGAGGCCCGCACCGATCGCCCTTCCC
AACAGTTGCGCAGCCTGAATGGCGAATGGGACGCGCCCTGTAGCGGCGCA
TTAAGCGCGGCGGGTGTGGTGGTTACGCGCAGCGTGACCGCTACACTTGC
CAGCGCCCTAGCGCCCGCTCCTTTCGCTTTCTTCCCTTCCTTTCTCGCCA
CGTTCGCCGGCTTTCCCCGTCAAGCTCTAAATCGGGGGCTCCCTTTAGGG
TTCCGATTTAGTGCTTTACGGCACCTCGACCCCAAAAAACTTGATTAGGG
TGATGGTTCACGTAGTGGGCCATCGCCCCGATAGACGGTTTTTCGCCCTT
TGACGCTGGAGTTCACGTTCCTCAATAGTGGACTCTTGTTCCAAACTGGA
ACAACACTCAACCCTATCTCGGTCTATTCTTTTGATTTATAAGGGATTTT
TCCGATTTCGGCCTATTGGTTAAAAAATGAGCTGATTTAACAAAAATTTA
ACGCGAATTTTAACAAAATATTAACGTTTATAATTTCAGGTGGCATCTTT
CGGGGAAATGTGCGCGGAACCCCTATTTGTTTATTTTTCTAAATACATTC
AAATATGTATCCGCTCATGAGACAATAACCCTGATAAATGCTTCAATAAT
ATTGAAAAAGGAAGAGTATGAGTATTCAACATTTCCGTGTCGCCCTTATT
CCCTTTTTTGCGGCATTTTGCCTTCCTGTTTTTGCTCACCCAGAAACGCT
GGTGAAAGTAAAAGATGCTGAAGATCAGTTGGGTGCACGAGTGGGTTACA
TCGAACTGGATCTCAATAGTGGTAAGATCCTTGAGAGTTTTCGCCCCGAA
GAACGTTTTCCAATGATGAGCACTTTTAAAGTTCTGCTATGTGGCGCGGT
ATTATCCCGTATTGACGCCGGGCAAGAGCAACTCGGTCGCCGCATACACT
ATTCTCAGAATGACTTGGTTGAGTACTCACCAGTCACAGAAAAGCATCTT
ACGGATGGCATGACAGTAAGAGAATTATGCAGTGCTGCCATAACCATGAG
TGATAACACTGCGGCCAACTTACTTCTGACAACGATCGGAGGACCGAAGG
AGCTAACCGCTTTTTTGCACAACATGGGGGATCATGTAACTCGCCTTGAT
CGTTGGGAACCGGAGCTGAATGAAGCCATACCAAACGACGAGCGTGACAC
CACGATGCCTGTAGTAATGGTAACAACGTTGCGCAAACTATTAACTGGCG
AACTACTTACTCTAGCTTCCCGGCAACAATTAATAGACTGGATGGAGGCG
GATAAAGTTGCAGGACCACTTCTGCGCTCGGCCCTTCCGGCTGGCTGGTT
TATTGCTGATAAATCTGGAGCCGGTGAGCGTGGGTCTCGCGGTATCATTG
CAGCACTGGGGCCAGATGGTAAGCCCTCCCGTATCGTAGTTATCTACACG
ACGGGGAGTCAGGCAACTATGGATGAACGAAATAGACAGATCGCTGAGAT
AGGTGCCTCACTGATTAAGCATTGGTAACTGTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGCGGTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGAAG

The resulting pAAV2.1-CMV-mTFEB-FLAG was then triple transfected in sub-confluent 293 cells along with the pAd-Helper and the pack2/1 or pack 2/9 packaging plasmids (Gao G. et al. J Virol. 2004, 78(12):6381-8.). The recombinant AAV2/1 or AAV2/9 vectors were purified by two rounds of CsCl.

Vector titers, expressed as genome copies (GC/mL), were assessed by both PCR quantification using TaqMan (Gao, G 2000) (Perkin-Elmer, Life and Analytical Sciences, Waltham, Mass.) and by dot blot analysis as described in Auricchio et al. (2001) Hum. Mol. Genet. 10(26):3075-81.

In the present invention AAV2.1 represents the plasmid coding for TFEB while AAV2/1 or AAV2/9 represents the virus containing the TFEB construct with serotype 1 or 9 capsid.

### Example 2

### TFEB Overexpression and Amelioration of PD Pathology

Experiments in this Example demonstrate that overexpression of TFEB by intramuscular injection of AAV2/1-CMV-mTFEB results in clearance of glycogen storages and amelioration of muscular pathology in Pompe disease models.

Sequence of the pAAV2.1 CMV-mTFEB Plasmid from 3′ITR to 5′ITR (SEQ ID NO: 7):

CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACGTAGCCATGCTCTAGGAAGATCGGAATTCGCCC
TTAAGCTAGCTAGTTATTAATAGTAATCAATTACGGGGTCATTAGTTCAT
AGCCCATATATGGAGTTCCGCGTTACATAACTTACGGTAAATGGCCCGCC
TGGCTGACCGCCCAACGACCCCCGCCCATTGACGTCAATAATGACGTATG
TTCCCATAGTAACGCCAATAGGGACTTTCCATTGACGTCAATGGGTGGAG
TATTTACGGTAAACTGCCCACTTGGCAGTACATCAAGTGTATCATATGCC
AAGTACGCCCCCTATTGACGTCAATGACGGTAAATGGCCCGCCTGGCATT
ATGCCCAGTACATGACCTTATGGGACTTTCCTACTTGGCAGTACATCTAC
GTATTAGTCATCGCTATTACCATGGTGATGCGGTTTTGGCAGTACATCAA
TGGGCGTGGATAGCGGTTTGACTCACGGGGATTTCCAAGTCTCCACCCCA
TTGACGTCAATGGGAGTTTGTTTTGGCACCAAAATCAACGGGACTTTCCA
AAATGTCGTAACAACTCCGCCCCATTGACGCAAATGGGCGGTAGGCGTGT
ACGGTGGGAGGTCTATATAAGCAGAGCTGGTTTAGTGAACCGTCAGATCC
TGCAGAAGTTGGTCGTGAGGCACTGGGCAG[GTAAGTATCAAGGTTACAA
GACAGGTTTAAGGAGACCAATAGAAACTGGGCTTGTCGAGACAGAGAAGA
CTCTTGCGTTTCTGATAGGCACCTATTGGTCTTACTGACATCCACTTTGC
CTTTCTCTCCACAG]GTGTCCAGGCGGCCGCATGGCTCAGCTCGCTCAGT
GGTCTTGGGCAAATCCCTTCTGCCCGGACTCAGTTTCTCCTTGTGCACAA
TGGGAGCAACCATACTTATGCCAGCCTGTGCTTAAAGACTACGAAGATGA
TGAATACTTCATGGGCCTGTCTCCCCTCGACTACAGGGAGCCCGAACCAA
CAGCTGCCATGGCGTCACGCATCGGGCTGCGCATGCAGCTCATGCGGGAG
CAGGCCCAGCAGGAGGAGCAGCGAGAGCGCATGCAGCAGCAGGCTGTCAT
GCATTATATGCAACAGCAGCAGCAGCAGCAGCAGCAGCTGGGTGGGCCCC
CCACCCCAGCCATCAACACCCCTGTCCACTTCCAGTCGCCCCCGCCTGTG
CCCGGGGAGGTGCTGAAGGTGCAGTCCTACCTGGAGAACCCCACCTCCTA
CCACCTGCAACAGTCCCAGCATCAGAAGGTTCGGGAGTATCTGTCTGAGA
CCTATGGGAACAAGTTTGCTGCCCACGTGAGCCCAGCCCAAGGTTCCCCG
AAGCCTGCCCCAGCAGCATCCCCAGGGGTGCGGGCTGGACACGTACTGTC
CACCTCGGCCGGCAACAGTGCTCCCAACAGTCCCATGGCCATGCTACATA
TCAGCTCCAACCCCGAGAAAGAGTTTGATGATGTCATTGACAACATTATG
CGCCTGGACAGCGTGCTGGGCTACATCAACCCTGAGATGCAGATGCCTAA
CACGCTGCCCCTGTCTAGCAGCCACCTGAACGTGTACAGCGGTGACCCCC
AGGTCACAGCCTCCATGGTGGGTGTCACCAGCAGCTCCTGCCCTGCCGAC
CTGACTCAGAAGCGAGAGCTAACAGATGCTGAGAGCAGAGCCCTGGCCAA
GGAGCGGCAGAAGAAAGACAATCACAACCTAATTGAGAGAAGACGCAGGT
TCAACATCAATGACCGGATCAAGGAGCTGGGAATGCTGATCCCCAAGGCC
AACGACCTGGACGTGCGCTGGAACAAAGGCACCATCCTCAAGGCCTCTGT
GGATTACATCCGGAGGATGCAGAAGGACCTGCAGAAGTCCCGGGAGCTGG
AGAACCACTCCCGGCGCCTGGAGATGACTAACAAGCAGCTCTGGCTCCGC
ATCCAGGAGCTGGAGATGCAGGCACGCGTGCACGGCCTCCCCACCACCTC
GCCGTCGGGTGTGAATATGGCCGAGCTGGCCCAGCAGGTGGTGAAGCAAG
AGTTGCCCAGTGAGGATGGCCCAGGGGAGGCGCTGATGCTGGGGCCTGAG
GTCCCTGAGCCTGAGCAAATGCCGGCTCTTCCTCCCCAGGCTCCGCTGCC
CTCGGCCGCCCAGCCACAGTCTCCGTTCCATCACCTGGACTTCAGCCATG
GCCTGAGCTTTGGGGGTGGGGGCGACGAGGGGCCCACAGGTTACCCCGAT
ACCCTGGGGACAGAGCACGGCTCCCCATTCCCCAACCTGTCCAAGAAGGA
TCTGGACTTAATGCTCCTAGATGACTCCCTGCTCCCCCTGGCCTCTGACC
CCCTCTTTTCTACCATGTCTCCTGAGGCCTCCAAGGCCAGCAGCCGCCGG
AGCAGCTTCAGCATGGAGGAGGGTGA(TGTTCTGGGATCCCGGGCTGACT
ACAAAGACCATGACGGTGATTATAAAGATCATGACATCGACTACAAGGAT
GACGATGACAAGTAG)TGAAAGCTTGGATCCAATCAACCTCTGGATTACA
AAATTTGTGAAAGATTGACTGGTATTCTTAACTATGTTGCTCCTTTTACG
CTATGTGGATACGCTGCTTTAATGCCTTTGTATCATGCTATTGCTTCCCG
TATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGTG
TTTGCTGACGCAACCCCCACTGGTTGGGGCATTGCCACCACCTGTCAGCT
CCTTTCCGGGACTTTCGCTTTCCCCCTCCCTATTGCCACGGCGGAACTCA
TCGCCGCCTGCCTTGCCCGCTGCTGGACAGGGGCTCGGCTGTTGGGCACT
GACAATTCCGTGGTGTTGTCGGGGAAGCTGACGTCCTTTCCATGGCTGCT
CGCCTGTGTTGCCACCTGGATTCTGCGCGGGACGTCCTTCTGCTACGTCC
CTTCGGCCCTCAATCCAGCGGACCTTCCTTCCCGCGGCCTGCTGCCGGCT
CTGCGGCCTCTTCCGCGTCTTCGAGATCTGCCTCGACTGTGCCTTCTAGT
TGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGAC
AGCAAGGGGGAGGATTGGGAAGACAATAGCAGGCATGCTGGGGACTCGAG
TTAAGGGCGAATTCCCGATTAGGATCTTCCTAGAGCATGGCTACGTAGAT
AAGTAGCATGGCGGGTTAATCATTAACTACAAGGAACCCCTAGTGATGGA
GTTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGAG
CGAGCGCGCAG

Bold nucleotides: 3′ITR and 5′ITR sequence
Italic nucleotides: CMV promoter sequence
Nucleotides in brackets [ . . . ]: SV40 intron sequence
Underlined nucleotides: mTFEB sequence
Nucleotides in parentheses ( . . . ): FLAG sequence
Bold Underlined nucleotides: WPRE sequence
Double underlined nucleotides: BGH polyA sequence

One-month old GAA−/− mice received a direct intramuscular injection of an AAV2/1-CMV-mTFEB vector in three sites of a single muscle, i.e., the right gastrocnemius. As a control, the mice received injections with either AAV2/1-EGFP vector or vehicle PBS (Phosphate Buffer Saline) alone into the contralateral muscle. The animals were sacrificed 45 days after injection, to allow maximal and sustained expression of the vector, and their muscles were analyzed.

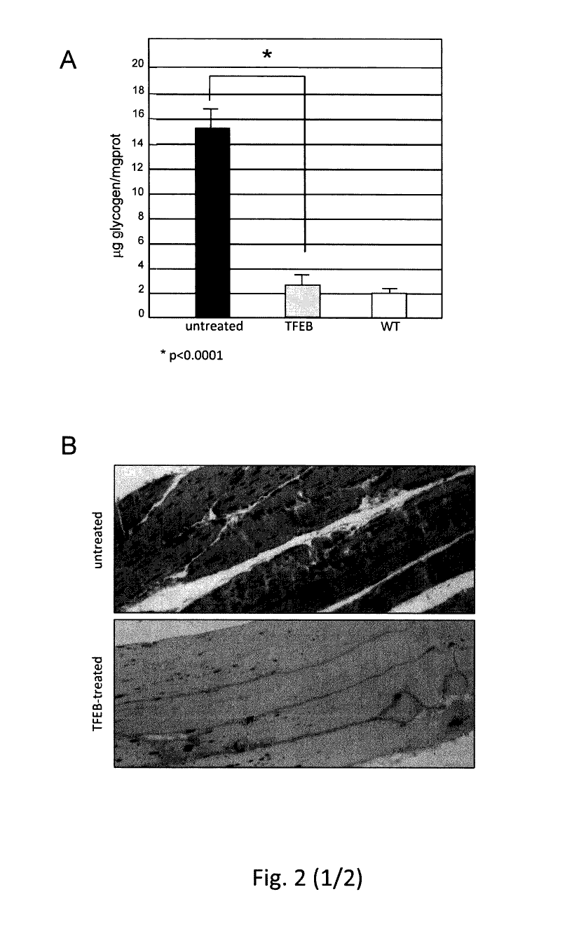

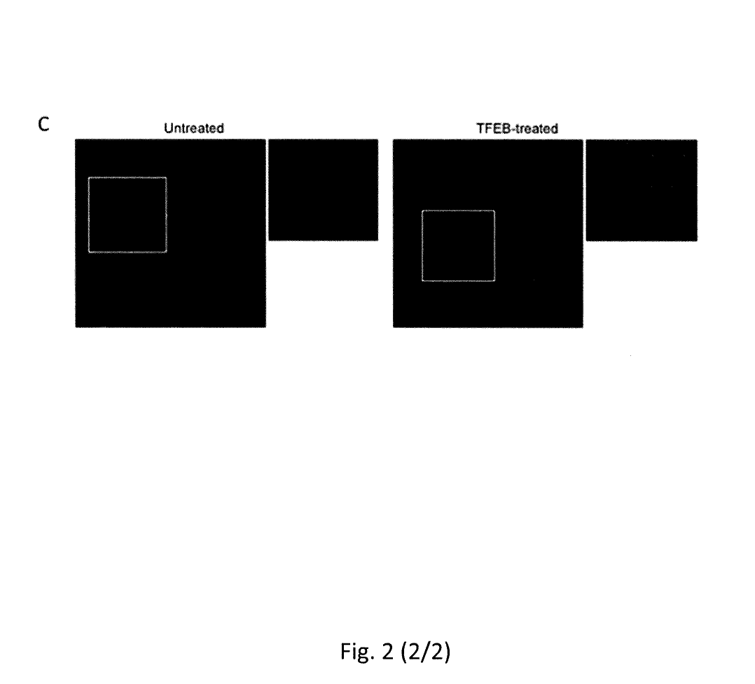

The average levels of TFEB expression, analyzed by real-time (RT)-PCR, were 10-fold higher in the AAV2/1-CMV-mTFEB-injected muscles compared to controls. Glycogen levels increased in muscles of mice treated with controls (15.48±1.80 μg glycogen/mg protein) as compared to glycogen levels in wild-type mice (2.01±0.70 μg glycogen/mg protein). On the contrary, glycogen levels decreased significantly (2.8±0.88 μg glycogen/mg protein, p=0.0001) in gastrocnemia muscles of mice treated with TFEB, indicating near-complete clearance of pathological glycogen stores. Exemplary results are depicted in FIG. 2A.

TFEB overexpression also resulted in the attenuation of the typical pathology of PD muscles. PAS staining of TFEB injected muscles showed a reduction of the punctate staining corresponding to lysosomal glycogen stores (glycogenosomes) as shown in FIG. 2B and also a reduction of LAMP1 vesicles as shown in FIG. 2C.

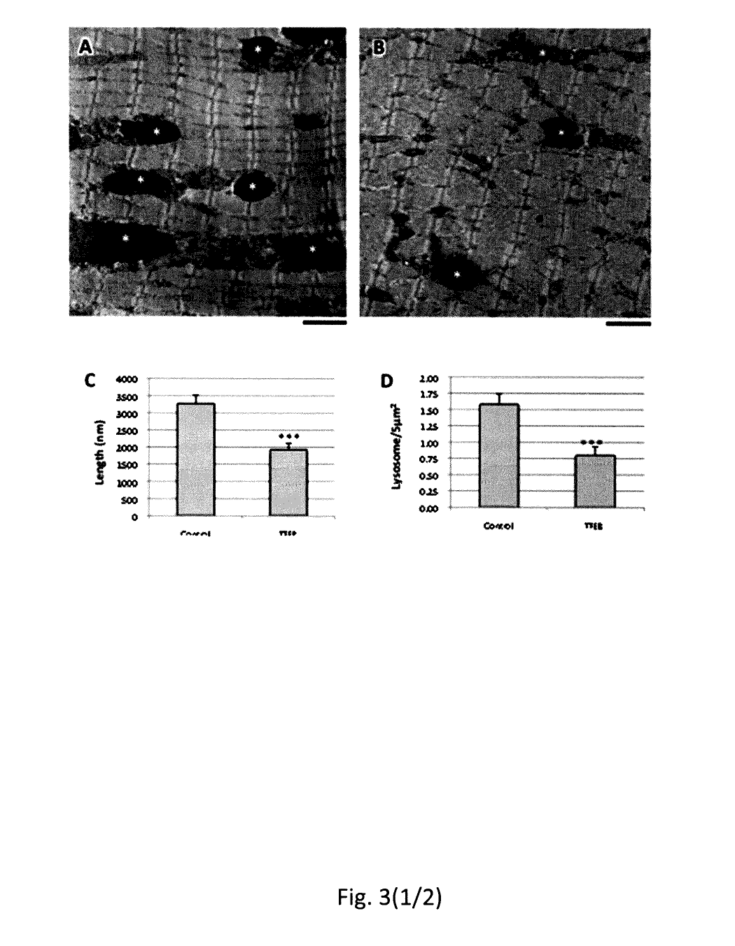

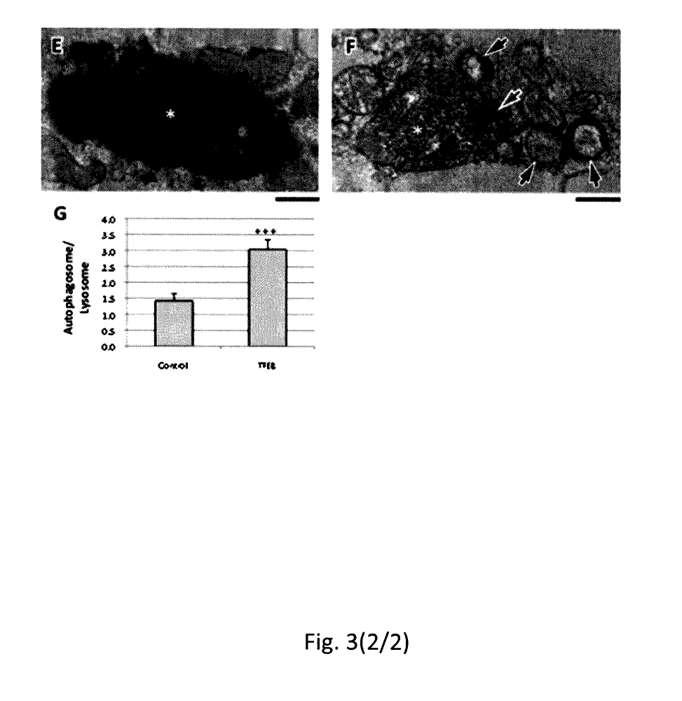

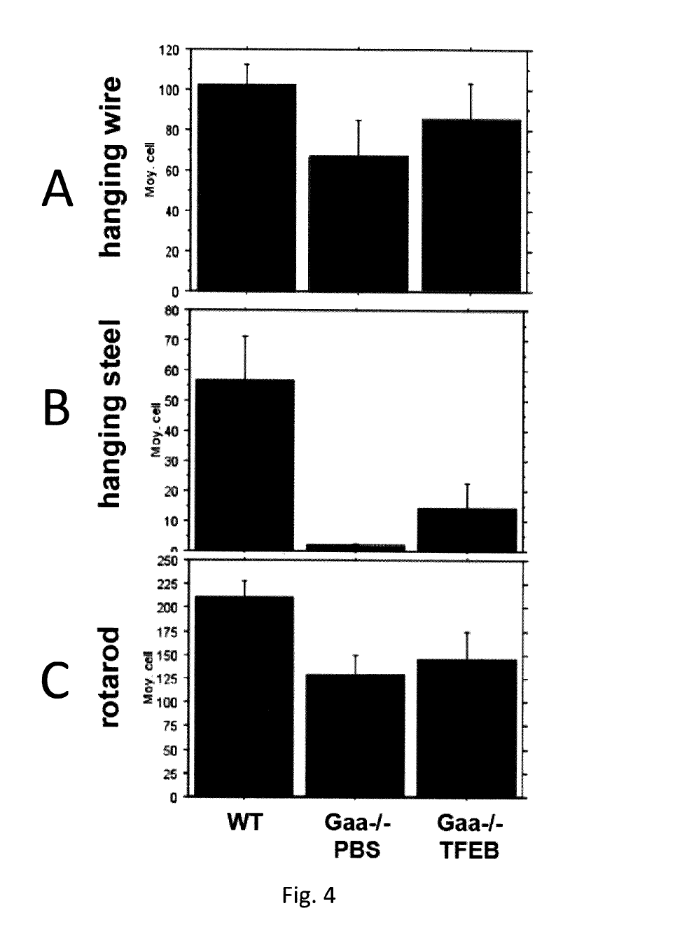

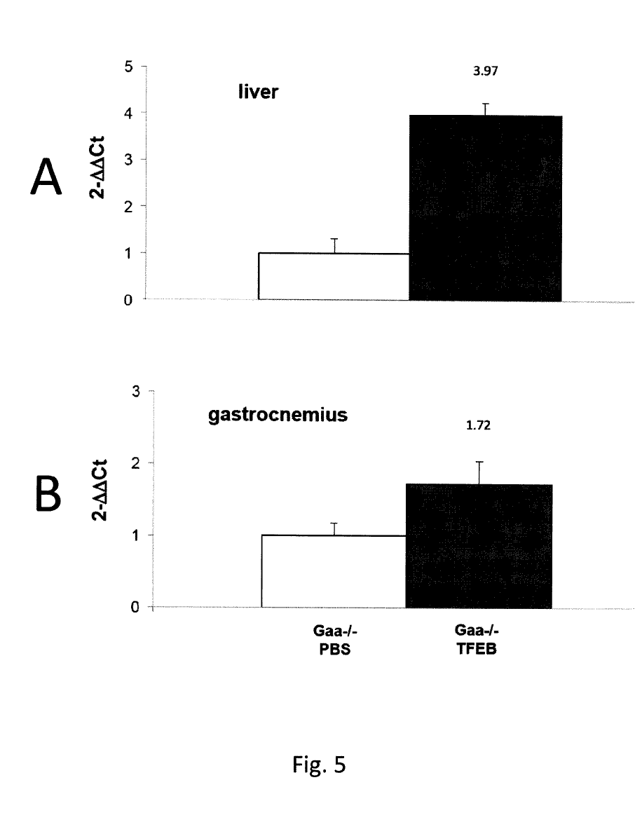

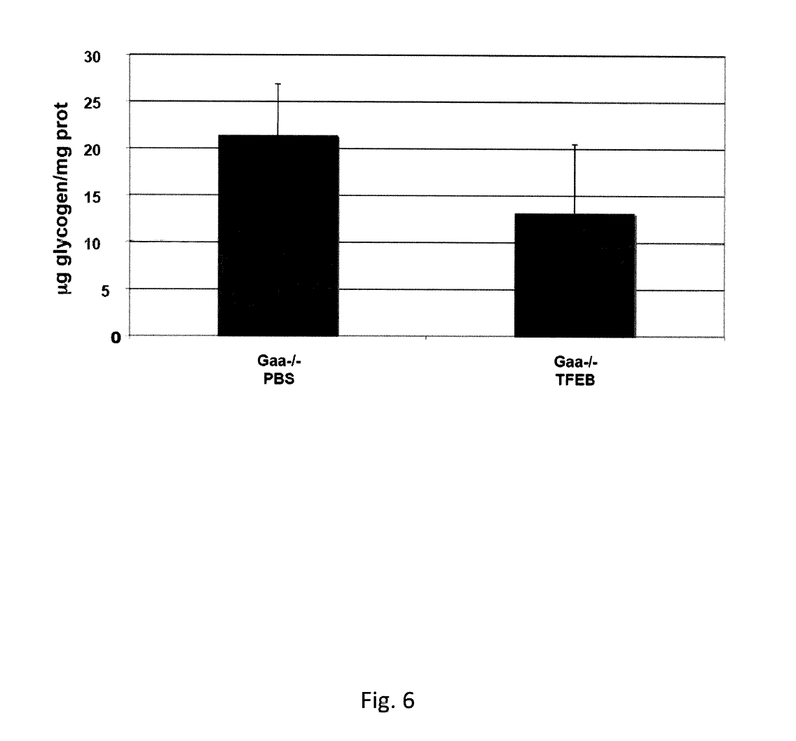

EM analysis was conducted from treated and untreated gastrocnemia to determine the ultrastructural changes induced by TFEB overexpression. In the untreated muscles the ultrastructural analysis showed the typical abnormalities of PD, with extensive disruption of the contractile apparatus due to the presence of multiple large lysosome-like structures densely filled by glycogen as marked by the asterisk in FIG. 3A.

TFEB overexpression resulted in a significant improvement of muscle fiber ultrastructure. A clear reduction in the size and number of glycogen-containing lysosomes detected in thin sections was observed as shown in FIG. 3B, supported by morphometric analysis as shown in FIGS. 3C and 3D. The large lysosome-like organelles packed with the electron-dense glycogen particles as were seen in untreated fibers, as marked by the asterisk in FIG. 3E, showed a significantly looser organization of glycogen in their interior in TFEB-treated muscles as marked by the asterisk in FIG. 3F.

An increased number of autophagosomes in close proximity to glycogen-containing organelles were also observed as shown by black arrows in FIG. 3F. Importantly, some autophagosomes contained glycogen particles as well, most likely directly derived from the cytosol. In addition, lysosomal structures frequently contained remnants of other intracellular organelles in their lumen as shown by white arrow in FIG. 3F, indicating the activation of their fusion with neighboring autophagosomes. The increase in the number of autophagosomes flanking the lysosomal structures in TFEB-treated muscle was confirmed by morphometric analysis as shown in FIG. 3G. Thus, the ultrastructural analysis indicated that the decrease in glycogen stores and the reduction of the number and size of glycogen-containing lysosomes was mediated by activation of autophagy and stimulation of the fusion of autophagosomes with lysosomes.

Overall, the data in this Example indicates that TFEB overexpression by intramuscular injection is able to significantly rescue glycogen storage and morphological abnormalities.

### Example 3

### Systemic Injection of AAV2/9-CMV-mTFEB Results in Decrease of Glycogen Stores

The authors have tested the effects of mTFEB systemic delivery in PD (Gaa−/−) mice. Six one-month-old Gaa−/− mice were injected with 1×10¹² gc/mouse AAV2/9-CMV-mTFEB vector via retro-orbital administration.

At the age of 2.5 months the animals were examined with behavioral tests (hanging wire, hanging steel, rotarod), and sacrificed to measure TFEB expression levels and glycogen content in muscles (gastrocnemii).

In all behavioral tests, both AAV2/9-CMV-mTFEB-treated and untreated animals showed impaired performance compared to wild-type animals (FIG. 4). However, TFEB-treated animals showed a trend towards improved performance, compared to untreated animals, suggesting a beneficial effect of TFEB overexpression on mice locomotor activity.

The expression levels of TFEB, analyzed by real-time PCR, were evaluated in liver and gastrocnemii of the treated mice. The analysis showed an increase of approximately 4 fold in liver (3,97±0,27) and of approximately 2 fold in gastrocnemius (1,72±0,32) in TFEB-injected mice, compared to their relative controls (FIG. 5).

In TFEB-treated animals gastrocnemii the authors observed decreased glycogen levels (FIG. 6), compared to untreated animals. These results suggest that TFEB overexpression after systemic delivery of AAV2/9-CMV-mTFEB results in improved clearance of substrate stores, as observed in animals treated with intra-muscular injection.

### Materials and Methods

### Animals

GAA−/− mice (KO PD mouse model) obtained by insertion of neo into the Gaa gene exon 6 [Raben et al, 1998] was purchased from Charles River Laboratories (Wilmington, Mass.). Animal studies were performed according to the European Union Directive 86/609, regarding the protection of animals used for experimental purposes. Every procedure on the mice was performed with the aim of ensuring that discomfort, distress, pain, and injury would be minimal. Mice were euthanized following avertin anesthesia by cervical dislocation

### Intra-Muscular Injection of AAV-TFEB

Six 1-month-old GAA−/− mice were injected with a total dose of 10¹¹ GC/muscle of AAV2/1-CMV-mTFEB vector preparation into 3 different sites of the right gastrocnemius muscle (3 injections of 30 μl each) using a 100 μl Hamilton syringe. Equivalent doses of AAV2/1CMV-EGFP or equal volumes of PBS were injected into the contralateral muscles for comparison. The animals were sacrificed 45 days after injection, perfused with PBS, and their muscles collected and were analyzed. The gastrocnemii were isolated and samples for biochemical analysis, light and immunofluorescence microscopy, and for electron microscopy (EM) were obtained. The levels of expression of TFEB were tested by RT=PCR.

### Systemic Injection of AAV-TFEB

Six one-month-old Gaa−/− mice were injected with 1×10¹² GC/mouse AAV2/9 CMV-mTFEB vector via retro-orbital administration. Equivalent doses of AAV2/9 CMV-eGFP or equal volumes of PBS alone were systemically injected as control. The animals were sacrificed 45 days after injection, perfused with PBS, and their organs were collected. The samples for biochemical analysis, light and immunofluorescence microscopy, and for electron microscopy (EM) were obtained. The levels of expression of TFEB were tested by RT-PCR.

### Glycogen Assay in Muscles

Glycogen concentration in muscles was assayed by measuring the amount of glucose released from a boiled tissue homogenate after digestion with Aspergillus niger amyloglucosidase as described in Raben et al. (2003) Molecular Genetics and Metabolism, Vol. 80, No. 1-2: 159-169 or by using a commercial kit (BioVision, Milpitas, Calif., USA).

Tissue lysates prepared by homogenization of tissue in H₂O were heat denatured at 99° C. for 10 minutes and centrifuged for 10 minutes at 4° C. Supernatants were incubated in duplicate with or without 10 μL of 800 U/mL amyloglucosidase for 1 hour at 37° C. The reactions were stopped by heat inactivation at 99° C. for 10 minutes. Glycogen from bovine liver (Sigma-Aldrich, St Louis, Mo., USA) hydrolyzed in the same conditions was used to generate a standard curve. Samples were centrifuged and the glucose level in the supernatant was determined using Glucose Assay Reagent (Sigma-Aldrich) according to the manufacturer's instructions.

Protein levels were measured in lysates (before denaturing) using the Biorad Protein Assay Kit according to the manufacturer's instructions. Data were expressed as micrograms of glycogen/milligram of protein (mg glycogen/mg protein).

### Period Acid Schiff (PAS) Staining of Muscles and Immunofluorescence Analysis of LAMP 1

Tissues were fixed in 10% formalin and embedded in paraffin. Cryostat sections were obtained and stained with HE and periodic acid-Schiff (PAS) by standard methods. For immunofluorescence analysis of LAMP1 the tissues were fixed in 4% PFA for 24 h at 4° C., embedded in paraffin (Sigma-Aldrich), dehydrated with a 70-100% ethanol gradient and serial 7 mm sections were obtained. Immunofluorescence analysis was performed as previously described in Settembre et al. (2007) “Systemic inflammation and neurodegeneration in a mouse model of multiple sulfatase deficiency”, PNAS 104:4506-11.

Serial sections were treated with xylene to remove paraffin, rehydrated, and treated for 15 minutes in a microwave oven with 0.05 mol/L glycine-HCl (pH 3.5) for antigen retrieval. The specimens were incubated for 1 h with blocking solution (PBS, 0.2% Tween-20) and 10% goat normal serum (Sigma-Aldrich) before incubation over night with the specific primary antibody. The antibodies used were LAMP1 (rabbit polyclonal 1:300; Sigma) and FLAG M2 (mouse monoclonal 1:300 Sigma). After washing, sections were incubated for 40 min with secondary antibody, purchased from Molecular Probes (Invitrogen, CA, USA). Stained sections were subsequently mounted with Vectashield with DAPI (Vector Laboratories, CA, USA). Images were taken by using a fluorescence microscope Zeiss (Thornwood, N.Y.) Axioplan 2 integrated with the AxioCam MR camera.

### Electron Microscopy

Small pieces of muscle tissue were dissected from GAA−/− mice injected with either AAV-TFEB (FIGS. 3B and 3F) or control AAV-EGFP (FIGS. 3A and 3E), fixed in 1% glutaraldehyde in 0.2 M HEPES buffer, post-fixed in uranyl acetate and in OsO₄. After dehydration through a graded series of ethanol and propilenoxide, the cells were embedded in the Epoxy resin (Epon 812, Sigma-Aldrich, St. Louis, Mo., USA) and polymerized at 60° C. for 72 h. From each sample, thin sections were cut with a Leica EM UC6 ultramicrotome (Leica Mycosystems, Vienna, Austria). EM images were acquired from thin sections using a FEI Tecnai-12 electron microscope (FEI, Eindhoven, Netherlands) equipped with a VELETTA CCD digital camera (Soft Imaging Systems GmbH, Munster, Germany). Quantification of the number of lysosome-like organelles and their dimensions as well as the number of autophagosomes was performed using the iTEM software (Soft Imaging Systems GmbH, Munster, Germany) in 50 fields (of 5 μm² dimensions) distributed randomly through the thin sections containing different fibers.

Wilcoxon rank sum test was used for comparison of median values. For all statistical analysis, Student's t-test and 95% confidence intervals (error bars; 1.96*SE) were calculated in Excel. Differences were considered significant at p<0.05.

### TFEB Expression Analyzed by Real-Time (RT)-PCR

To evaluate TFEB expression levels in tissue, total RNA was extracted using RNeasy kit Qiagen (Hilden, Germany) according to the manufacturer's instructions. One □ g of RNA was used to prepare the relevant cDNA with SuperScript II First Strand Synthesis System (Invitrogen, Carlsbad, Calif.). Real time PCR was performed using the SYBR-green PCR master mix (Applied Biosystems, Foster City, Calif.) on a LightCycler 480 instrument (Roche, Basel, Switzerland) and data were represented as DDCt. TFEB Fw primer: 5′-gcagaagaaagacaatcacaacc-3′ (SEQ ID NO: 8); TFEB Rv primer: 5′-gccttggggatcagcatt-3′(SEQ ID NO: 9).

### Behavioral Analysis

For the behavioral procedures both treated and untreated mice underwent to the following tests: hanging wire, hanging steel and rotarod tests, according to published procedures (Raben N. et al, J Biol Chem. 1998, 273(30):19086-92.; Sidman R L et al, J Neuropathol Exp Neurol. 2008, 67(8):803-18).

### INCORPORATION OF REFERENCES

All publications and patent documents cited in this application are incorporated by reference in their entirety to the same extent as if the contents of each individual publication or patent document were incorporated herein.

1.-31. (canceled)

32. A method of treating a glycogen storage disease comprising a step of delivering a nucleic acid encoding a transcription factor EB (TFEB) gene into a subject in need of treatment.

33. (canceled)

34. The method according to claim 33 wherein the glycogen storage disease is selected from the group consisting of: GSD type Ia (Von Gierke disease), GSD type I non-a (various subtypes), GSD type II (Pompe disease), GSD type IIb (Danon disease), GSD type III (Cori's disease or Forbes' disease), GSD type IV (Andersen disease), GSD type V (McArdle disease), GSD type VI (Hers' disease), GSD type VII (Tarui's disease), GSD type IX, GSD type XI (Fanconi-Bickel syndrome), GSD type XII (Red cell aldolase deficiency), GSD type XIII and GSD type 0.

35. The method according to claim 32 wherein the glycogen storage disease is Pompe disease.

36. The method according to claim 32 wherein the glycogen storage disease is Danon disease.

37. The method according to claim 32, wherein the nucleic acid encoding the TFEB gene is delivered to a target tissue that contains accumulated glycogen.

38.-44. (canceled)

45. The method according to claim 32, wherein the nucleic acid is a viral vector.

46. The method according to claim 45, wherein the viral vector is an adeno-associated virus (AAV) vector.

47. The method according to claim 46, wherein the AAV vector is selected from the group consisting of AAV1, AAV2, AAV5, AAV6, AAV7, AAV8, AAV9, and combinations thereof.

48. The method according to claim 47, wherein the AAV vector is an AAV1, AAV2 or AAV9 vector.

49. The method according to claim 46, wherein the AAV vector is a chimeric and/or pseudotyped vector.

50. The method according to claim 32, wherein the nucleic acid further comprises a tissue specific promoter sequence that controls the expression of the TFEB gene.

51. The method according to claim 50, wherein the tissue specific promoter sequence is a muscle specific promoter sequence, preferably it is the MCK promoter sequence consisting of SEQ ID NO: 3.

52. The method according to claim 50, wherein the tissue specific promoter sequence is a liver specific promoter sequence, preferably it is the PEPCK promoter sequence consisting of SEQ ID NO: 4.

53. The method according to claim 32, wherein the TFEB gene comprises a nucleotide sequence at least 60% identical to SEQ ID NO: 1.

54. The method according to claim 32, wherein the TFEB gene comprises a nucleotide sequence at least 80% identical to SEQ ID NO: 1.

55. The method according to claim 32, wherein the TFEB gene comprises a nucleotide sequence of SEQ ID NO: 1.

56. The method according to claim 32, wherein the TFEB gene comprises a nucleotide sequence encoding an amino acid sequence at least 80% identical to SEQ ID NO: 2.

57. The method according to claim 32, wherein the TFEB gene comprises a nucleotide sequence encoding an amino acid sequence at least 90% identical to SEQ ID NO: 2.

58. The method according to claim 32, wherein the TFEB gene comprises a nucleotide sequence encoding an amino acid sequence of SEQ ID NO: 2.

59.-60. (canceled)

61. A method of treating a glycogen storage disease comprising a step of administering a nucleic acid encoding a transcription factor EB (TFEB) gene into a subject in need of treatment such that the glycogen storage in muscles and/or liver is reduced in intensity, severity, or frequency, or has delayed onset.

62. (canceled)

63. The method according to claim 61 wherein the glycogen storage disease is selected from the group consisting of: GSD type Ia (Von Gierke disease), GSD type I non-a (various subtypes), GSD type II (Pompe disease), GSD type IIb (Danon disease), GSD type III (Cori's disease or Forbes' disease), GSD type IV (Andersen disease), GSD type V (McArdle disease), GSD type VI (Hers' disease), GSD type VII (Tarui's disease), GSD type IX, GSD type XI (Fanconi-Bickel syndrome), GSD type XII (Red cell aldolase deficiency), GSD type XIII and GSD type 0.

64. The method according to claim 61, wherein the glycogen storage disease is Pompe disease.

65. The method according to claim 61, wherein the glycogen storage disease is Danon disease.

66.-68. (canceled)

69. The method according to claim 61, wherein the nucleic acid is an expression vector selected in the group consisting of: viral vector, plasmids, viral particles and phages.

70. The method according to claim 69, wherein the viral vector is selected from the group consisting of: adenoviral vectors, lentiviral vectors, retroviral vectors, adeno associated vectors (AAV) and naked plasmid DNA vectors.

71. The method according to claim 70, wherein the AAV vector is selected from the group consisting of AAV1, AAV2, AAV5, AAV6, AAV7, AAV8, AAV9, and combinations thereof.

72. The method according to claim 71, wherein the AAV vector is an AAV1, AAV2 or AAV9 vector.
